(19) United States
(12) Patent Application Publication
(10) Pub. No.: US 2025/0095337 A1
(43) Pub. Date: Mar. 20, 2025

(54) METHOD FOR CREATING MACHINE LEARNING MODEL FOR OUTPUTTING FEATURE MAP

(71) Applicants: Junya FUKUOKA, Nagasaki-shi, Nagasaki (JP); Wataru UEGAMI, Nagasaki-shi, Nagasaki (JP); NATIONAL INSTITUTE OF ADVANCED INDUSTRIAL SCIENCE AND TECHNOLOGY, Tokyo (JP)

(72) Inventors: Junya FUKUOKA, Nagasaki-shi, Nagasaki (JP); Wataru UEGAMI, Nagasaki-shi, Nagasaki (JP)

(73) Assignee: NATIONAL INSTITUTE OF ADVANCED INDUSTRIAL SCIENCE AND TECHNOLOGY, Tokyo (JP)

(21) Appl. No.: 18/580,323

(22) PCT Filed: Jul. 19, 2022
(86) PCT No.: PCT/JP2022/028099

§ 371 Date: Jan. 18, 2024

(30) Foreign Application Priority Data
Jul. 20, 2021 (JP) 2021-119842

(51) Int. Cl. G06V 10/774; G06T 7/00; G06V 10/26; G06V 10/762; G06V 10/764; G06V 10/77; G16H 30/40; G16H 50/20

(52) U.S. Cl. CPC G06V 10/774; G06T 7/0012; G06V 10/26; G06V 10/762; G06V 10/764; G06V 10/7715; G16H 30/40; G16H 50/20; G06T 2207/10081; G06T 2207/20081; G06T 2207/30061; G06V 2201/03

(57) ABSTRACT

This method for creating a machine learning model for outputting a feature map involves receiving a plurality of learning images, using an initial machine learning model to sort the plurality of learning images into respective initial clusters from among a plurality of initial clusters, resorting the plurality of initial clusters into a plurality of secondary clusters on the basis of the plurality of learning images as sorted into each of the plurality of initial clusters, and creating a m machine learning model by making the initial machine learning model learn the relationship between the plurality of initial clusters and the plurality of secondary clusters, the machine learning model being for sorting single inputted images into single secondary clusters from among the plurality of secondary clusters.

### TECHNICAL FIELD

The present invention relates to a method for creating a machine learning model for outputting a feature map, etc. The present invention also relates to a method of creating a feature map by using the created machine learning model, a method of estimating a state related to a disease of a subject by using the created feature map, a method of creating a machine learning model for classification, etc.

### BACKGROUND ART

Initiatives for predicting a disease of a subject by using a machine learning model are ongoing (e.g., Patent Literature 1).

### CITATION LIST

### Patent Literature

[PTL 1] Japanese National Phase PCT Laid-open Publication No. 2020-532025

### SUMMARY OF INVENTION

### Technical Problem

The inventors contemplated that a machine learning model that is capable of providing a meaningful output can be provided by fusing a machine learning model with human knowledge.

One of the objectives of the present invention is to provide a machine learning model that is capable of integrating human knowledge.

### Solution to Problem

The present invention provides, for example, the following items in one embodiment.

### (Item 1)

A method of creating a machine learning model comprising:
receiving a plurality of images for learning;
classifying each of the plurality of images for learning into a respective initial cluster of a plurality of initial clusters by using an initial machine learning model, the initial machine learning model being caused to at least learn to output a feature of an inputted image from the image;
reclassifying the plurality of initial clusters into a plurality of secondary clusters based on the plurality of images for classified learning into the respective plurality of initial clusters; and
creating a machine learning model by causing the initial machine learning model to learn a relationship between the plurality of initial clusters and the plurality of secondary clusters, the machine learning model classifying an inputted image into one of the plurality of secondary clusters.

### (Item 2)

The method 41 item 1, wherein the reclassifying comprises:
presenting the plurality of images for learning classified into the respective plurality of initial clusters to a user;
receiving a user input that associates each of the plurality of initial clusters to one of the plurality of secondary clusters; and
reclassifying the plurality of initial clusters into a plurality of secondary clusters based on the user input.

### (Item 3)

The method of item 2, wherein the plurality of secondary clusters are defined by the user.

### (Item 4)

The method of any one of items 1 to 3, wherein the plurality of secondary clusters are determined in accordance with a resolution of the plurality of images for learning.

### (Item 5)

The method of any one of items 1 to 4, wherein the plurality of images for learning comprise a plurality of partial images from fragmenting an image at a predefined resolution.

### (Item 6)

The method of any one of items 1 to 5, wherein the plurality of images for learning comprise an image for a pathological diagnosis.

### (Item 7)

The method of any one of items 1 to 6, wherein the plurality of images for learning comprises an image of tissue of a subject with interstitial pneumonia and an image of tissue of a subject without interstitial pneumonia.

### (Item 8)

The method of any one of items 1 to 7, further comprising repeating the receiving, the classifying, and the reclassifying of an image within at least one secondary cluster among the plurality of secondary clusters as the plurality of images for learning.

### (Item 9)

The method of any one of items 1 to 8, wherein the created machine learning model is used for outputting a feature map.

### (Item 10)

The method of any one of items 1 to 8, wherein the plurality of images for learning comprise a plurality of images of subjects with different diseases.

### (Item 11)

A method of creating a machine learning model, comprising:
receiving a plurality of images classified into at least one secondary cluster by a machine learning model created in accordance with the method of any one of items 1 to 10;
classifying each of the plurality of received images into a respective initial cluster of a plurality of initial clusters by using an initial machine learning model, the initial machine learning model being caused to at least learn to output a feature of an inputted image from the image;
reclassifying the plurality of initial clusters into a plurality of secondary clusters based on the plurality of received images classified into the respective plurality of initial clusters; and
creating a machine learning model by causing the initial machine learning model to learn a relationship between the plurality of initial clusters and the plurality of secondary clusters, the machine learning model classifying an inputted image into one of the plurality of secondary clusters.

### (Item 12)

A method of creating a feature map, comprising:
receiving a target image;
fragmenting the target image into a plurality of regional images;
classifying each of the plurality of regional images into a respective secondary cluster of the plurality of secondary clusters by inputting the plurality of regional images into a machine learning model created by the method of item 9; and
creating a feature map by separating each of the plurality of regional images in the target image in accordance with respective classifications.

### (Item 13)

The method of item 12, wherein the separating comprises coloring regional images belonging to the same classification among the plurality of regional images with the same color.

### (Item 14)

A method of estimating a state related to a disease of a subject, comprising:
obtaining a feature map created in accordance with the method of any one of items 12 to 13, the target image being an image of tissue of the subject; and
estimating a state related to a disease of the subject based on the feature map.

### (Item 15)

The method of item 14, wherein the estimating the state comprises estimating a type of interstitial pneumonia of the subject.

### (Item 16)

The method of item 14, wherein the estimating the state comprises estimating whether the subject has usual interstitial pneumonia.

### (Item 17)

The method of any one of items 14 to 16, wherein the estimating a state related to a disease of the subject based on the created feature map comprises:
calculating a frequency of each of the plurality of secondary clusters from the feature map; and
estimating a state related to the disease based on the frequency.

### (Item 18)

The method of any one of items 14 to 17, wherein creating the feature map comprises creating a plurality of feature maps, the plurality of feature maps having different resolutions from one another.

### (Item 19)

The method of item 18, wherein the estimating a state related to a disease based on the created feature map comprises:
calculating a frequency of each of the plurality of secondary clusters from each of the plurality of feature maps; and
estimating a state related to the disease based on the frequency.

### (Item 20)

The method of item 18 or 19, wherein the estimating a state related to a disease based on the created feature map comprises:
identifying an error in at least one of the plurality of feature maps by using the plurality of feature maps; and
estimating a state related to the disease based on at least one feature map excluding the at least one feature map in which an error has been identified.

### (Item 21)

The method of any one of items 14 to 20, further comprising:

analyzing survival time of the subject whose state related to the disease has been estimated based on the created feature map; and
identifying at least one secondary cluster contributing to the estimated state among a plurality of secondary clusters in the feature map.

### (Item 22)

A system for creating a machine learning model, comprising:
receiving means for receiving a plurality of images for learning;
classifying means for classifying each of the plurality of images for learning into a respective cluster of a plurality of initial clusters by using an initial machine learning model, the initial machine learning model being caused to at least learn to output a feature of an inputted image from the image;
reclassifying means for reclassifying the plurality of initial clusters into a plurality of secondary clusters based on the plurality of images for learning classified into the respective plurality of initial clusters; and
creating means for creating a machine learning model by causing the initial machine learning model to learn a relationship between the plurality of initial clusters and the plurality of secondary clusters, the machine learning model classifying an inputted image into one of the plurality of secondary clusters.

### (Item 22A)

The system of item 22, comprising a feature of one or more of the preceding items.

### (Item 23)

A program for creating a machine learning model, the program being executed in a computer system comprising a processing unit, the program causing the processing unit to execute processing comprising:
receiving a plurality of images for learning;
classifying each of the plurality of images for learning into a respective cluster of a plurality of initial clusters by using an initial machine learning model, the initial machine learning model being caused to at least learn to output a feature of an inputted image from the image;
reclassifying the plurality of initial clusters into a plurality of secondary clusters based on the plurality of images for learning classified into the respective plurality of initial clusters; and
creating a machine learning model by causing the initial machine learning model to learn a relationship between the plurality of initial clusters and the plurality the machine learning model of secondary clusters, classifying an inputted image into one of the plurality of secondary clusters.

### (Item 23A)

The program of item 23 comprising a feature of one or more of the preceding items.

### (Item 23B)

A computer readable storage medium for storing the program of item 23 or 23A.

### (Item 24)

A method of creating a machine learning model for classification, comprising:
receiving a plurality of data for learning;
classifying each of the plurality of data for learning into a respective cluster of a plurality of initial clusters by using an initial machine learning model, the initial machine learning model being caused to at least learn to output a feature of an inputted datum from the datum;
reclassifying the plurality of initial clusters into a plurality of secondary clusters based on the plurality of data for learning classified into the respective plurality of initial clusters; and
creating a machine learning model by causing the initial machine learning model to learn a relationship between the plurality of initial clusters and the plurality of secondary clusters, the machine learning model classifying an inputted datum into one of the plurality of secondary clusters.

### (Item 24A)

The method of item 24 comprising a feature of one or more of the preceding items.

### (Item 25)

A system for creating a machine learning model for classification, comprising:

receiving means for receiving a plurality of data for learning;
classifying means for classifying each of the plurality of data for learning into a respective cluster of a plurality of initial clusters by using an initial machine learning model, the initial machine learning model being caused to at least learn to output a feature of an inputted datum from the datum;
reclassifying means for reclassifying the plurality of initial clusters into a plurality of secondary clusters based on the plurality of data for learning classified into the respective plurality of initial clusters; and
creating means for creating a machine learning model by causing the initial machine learning model to learn a relationship between the plurality of initial clusters and the plurality of secondary clusters, the machine learning model classifying an inputted datum into one of the plurality of secondary clusters.

### (Item 25A)

The system of item 25 comprising a feature of one or more of the preceding items.

### (Item 26)

A program for creating a machine learning model for classification, the program being executed in a computer system comprising a processing unit, the program causing the processing unit to execute processing comprising:
receiving a plurality of data for learning;
classifying each of the plurality of data for learning into a respective cluster of a plurality of initial clusters by using an initial machine learning model, the initial machine learning model being caused to at least learn to output a feature of an inputted datum from the datum;
reclassifying the plurality of initial clusters into a plurality of secondary clusters based on the plurality of data for learning classified into the respective plurality of initial clusters; and
creating a machine learning model by causing the initial machine learning model to learn a relationship between the plurality of initial clusters and the plurality of secondary clusters, the machine learning model classifying an inputted datum into one of the plurality of secondary clusters.

### (Item 26A)

The program of item 26 comprising a feature of one or more of the preceding items.

### (Item 26B)

A computer readable storage medium for storing the program of item 26 or 26A.

### Advantageous Effects of Invention

The present invention can provide a machine learning model that is capable of integrating human knowledge. A feature map created by using such a machine learning model can reflect human knowledge. Use of such a feature map enables a state related to a disease of a subject to be estimated with high precision.

### BRIEF DESCRIPTION OF DRAWINGS

FIG. 1A Diagram showing an example of a flow for machine learning model that is capable of creating a integrating human knowledge

FIG. 1B Diagram showing an example of a plurality of images for learning classified into respective plurality of initial clusters

FIG. 1C Diagram showing an example of an image of tissue inputted into machine learning model 10 and an example of a feature map created in accordance with a classification outputted from machine learning model 10

FIG. 1D Diagram showing a specific example of the flow of FIG. 1A FIG. 2 Diagram showing an example of the configuration of system 100 for creating a machine learning model for outputting a feature map

FIG. 3A Diagram showing an example of the configuration of processing unit 120 in one embodiment

FIG. 3B Diagram showing an example of the configuration of processing unit 130 in another embodiment

FIG. 3C Diagram showing an example of the configuration of processing unit 140 in yet another embodiment

FIG. 4 Diagram showing an example of the configuration of terminal apparatus 300

FIG. 5 Flowchart showing an example of processing in system 100

FIG. 6 Flowchart showing another example of processing in system 100

FIG. 7 Flowchart showing another example of processing in system 100

FIG. 8 Diagram showing results of an Example

FIG. 9A Diagram showing results of an Example

FIG. 9B Diagram showing results of a Comparative Example

FIG. 10 Diagram showing results of an Example

FIG. 11 Diagram showing results of an Example

FIG. 12A Diagram showing results of an Example

FIG. 12B Diagram showing results of an Example

FIG. 13 Diagram showing image (a) of a cell marked with ink and a feature map created from the image

FIG. 14 Example of inputting a CT image of a lung into a machine learning model

### DESCRIPTION OF EMBODIMENTS

The present disclosure is described hereinafter. Throughout the entire specification, a singular expression should be understood as encompassing the concept thereof in the plural form, unless specifically noted otherwise. Thus, singular articles (e.g., “a”, “an”, “the”, etc. in case of English) should also be understood as encompassing the concept thereof in the plural form, unless specifically noted otherwise. The terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of contradiction, the present specification (including the definitions) takes precedence.

### Definition

As used herein, “subject” refers to any human or animal targeted by the technology of the invention.

As used herein, “disease” refers to a poor or disadvantaged state of a subject. “Disease” may be synonymously used with terms such as “disorder” (state with a normal function obstructed), “symptom” (abnormal state of a target), and “syndrome” (state with several symptoms manifested).

As used herein, “state” of a “subject” refers to the physical or mental status of the subject.

As used herein, “estimate a state” can be a concept including estimating a future state in addition to estimating a current state. Examples of “estimating a state related to a disease of a subject” includes estimating that a subject has some type of a specific disease, estimating that a subject does not have some type of a specific disease, estimating that a subject has at least one specific disease, estimating that a subject does not have at least one specific disease, estimating the type of at least one disease of a subject, estimating that the type of at least one disease of a subject is a specific type, estimating that the type of at least one disease of a subject is not a specific type, estimating the severity of at least one disease of a subject, estimating the severity of a specific type of at least one disease of a subject, etc.

As used herein, “feature map” refers to an image preparing from fragmenting an image into a plurality of regions and expressing regions with the same feature among the plurality of regions in the same manner. In one example, a feature map can be an image from coloring regions with the same feature among the plurality of regions with the same color.

As used herein, “image of tissue” refers to an image obtained from tissue harvested from the body of a subject. In one example, “image of tissue” can be a WSI (whole slide image). In one example, “image of tissue” can be an image obtained from tissue staining and/or an image obtained by immunohistological staining. In one example, the image can be a radiographic image obtained using an X-ray apparatus. In one example, “image of tissue” can be a microscopic image obtained by using a microscope. In this manner, “image of tissue” can be obtained by any means.

As used herein, “about” refers to a numerical range of ±10% from the numerical value that is described subsequent to “about”.

The embodiments of the invention are described hereinafter with reference to the drawings.

1. Flow for Creating a Machine Learning Model that is Capable of Integrating Human Knowledge

The inventors of the invention have developed a machine learning model that is capable of integrating human knowledge. Such a machine learning model can provide an output with a higher precision than that from an initial machine learning model because an output from the initial machine learning model is refined to be used for learning of the initial machine learning model in the creation stage thereof. In particular, a classification outputted from an initial machine learning model (so-called classifier) is refined through reclassification a by human, more preferably by a specialist or expert, whereby knowledge of the human, more preferably the specialist or expert, is integrated into a classification outputted from a machine learning model. For example, a classification outputted from a machine learning model can be a classification with histopathological meaning added thereto by a pathologist reclassifying a classification outputted from an initial learning model.

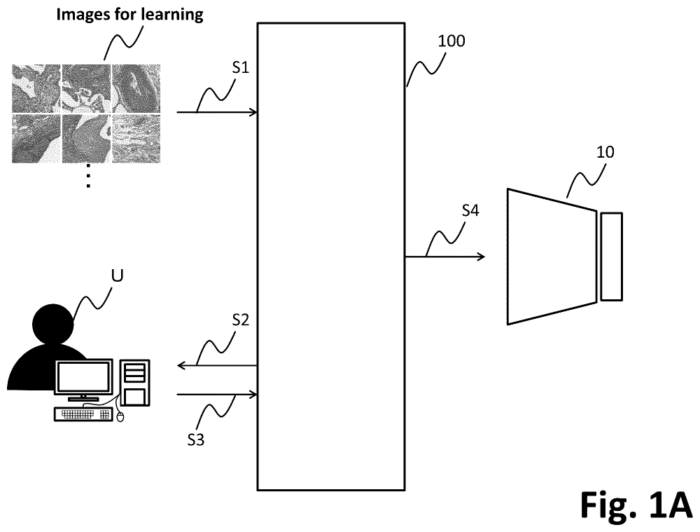

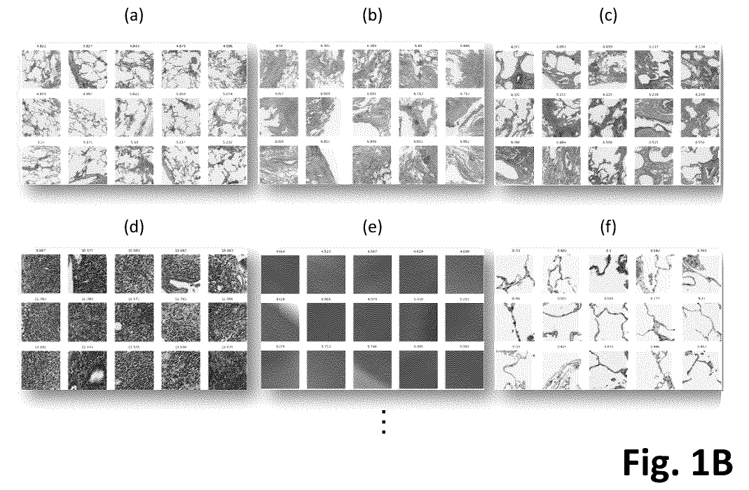

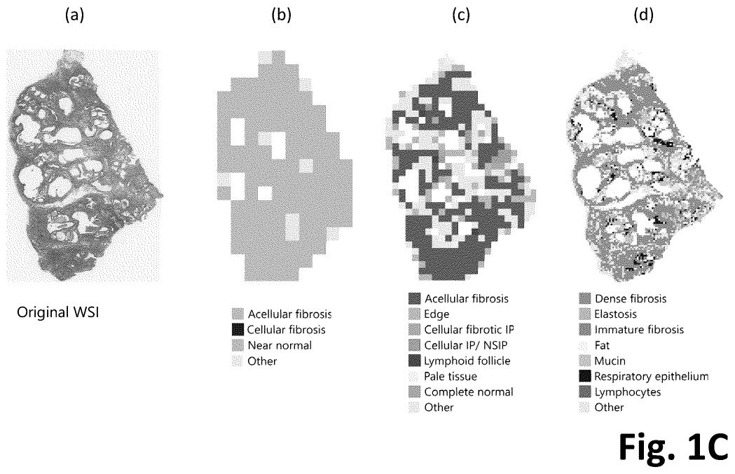

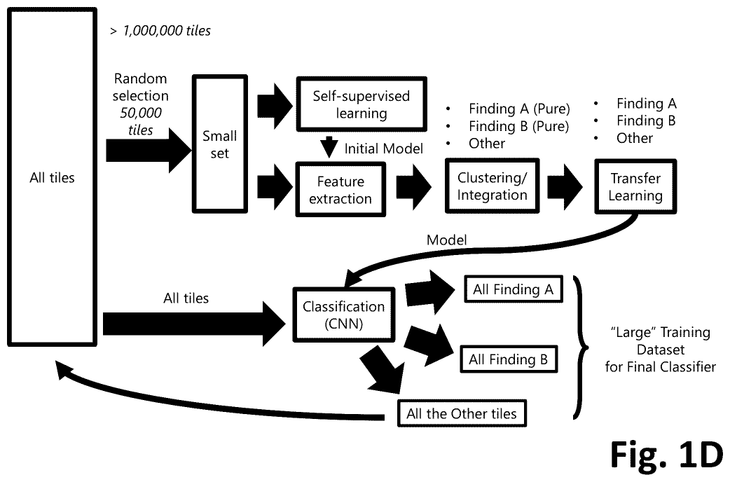

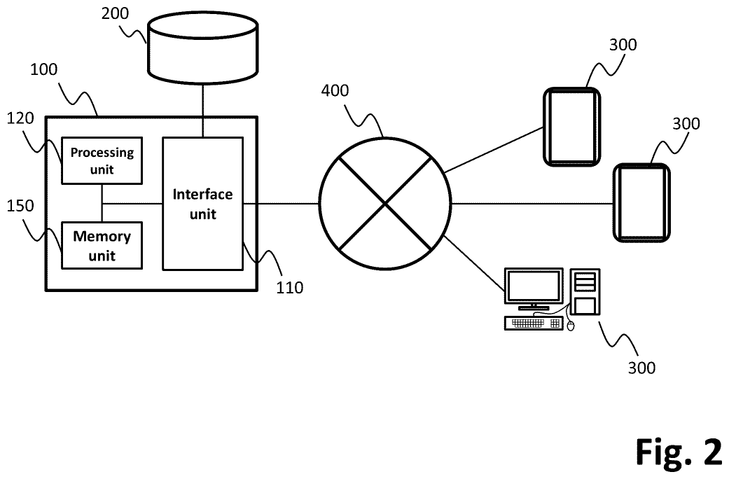

FIG. 1A shows an example of a flow for creating a machine learning model that is capable of integrating human knowledge.

At step S1, a plurality of images for learning are inputted into a system 100 for creating a machine learning model. In this example, a plurality of partial images from fragmenting a WSI (whole slide image) from tissue staining used in pathological diagnosis into a plurality of regions at a predefined resolution are used as a plurality of images for learning in order to create a machine learning model that is capable of outputting a histopathologically meaningful classification.

A plurality of images for learning can utilize any image in accordance with the application of the created machine learning model. For example, a plurality of partial images from fragmenting a radiographic image into a plurality of regions at a predefined resolution can be used as a plurality of images for learning in order to create a machine learning model that is capable of outputting a classification that is meaningful for radiographic diagnosis. For example, high resolution tomographic images or projectional X-ray images of the chest can be used as a plurality of images for learning in order to create a machine learning model that is capable of outputting a classification that is meaningful for a pathological classification of interstitial pneumonia. For example, images of a plurality of subjects having various diseases can be used as a plurality of images for learning in order to create a machine learning model that is capable of outputting classifications for various diseases. Specifically, images of various cancer cells can be used as a plurality of images for learning in order to create a machine learning model that is capable of outputting classifications for various cancers.

As described below, a plurality of images for learning may be a plurality of images grouped in accordance with classifications outputted from a machine learning model created by the system 100. For example, a plurality of images for learning may be images classified into the cluster of “Others” by a machine learning model. As described below, a plurality of images for learning may be a plurality of images grouped in accordance with reclassification by a user U. For example, a plurality of images for learning may be images reclassified into the cluster of “Others” by the user U.

The embodiment of inputting a plurality of images for learning into the system 100 is not limited. A plurality of images for learning can be inputted into the system 100 in any manner. For example, a plurality of images for learning may be configured to be inputted into the system 100 through a network (e.g., Internet, LAN, etc.), e.g., inputted into the system 100 through a storage medium that can be connected to the system 100 or inputted into the system 100 through an image acquisition apparatus that the system 100 can comprise.

The plurality of images for learning that have been inputted are inputted into initial machine learning model in the system 100. The initial machine learning model is caused to at least learn to output a feature of an inputted image from the image. The image can be classified into one of a plurality of initial clusters by clustering outputted features.

When a plurality of images for learning are inputted into an initial machine learning model, a feature of each of the plurality of images for learning is outputted, and each of the features is clustered, whereby each of the plurality of images for learning is classified into a respective e initial cluster of a plurality of initial clusters. An initial cluster classified in this manner is classified based on a feature of an image, which may not be a meaningful classification. To refine such an initial cluster, an output from an initial machine learning model needs to be reclassified.

At step S2, a plurality of images for learning classified into respective initial clusters are presented to the user U. The user U is preferably, for example, a specialist or expert such as a pathologist. For example, a plurality of images for learning classified into respective plurality of initial clusters are presented to the user U as shown in FIG. 1B.

While FIG. 1B shows six initial clusters (a) to (f), the number of initial clusters is not limited thereto. An initial cluster can comprise any number of clusters. As shown in FIG. 1B, images for learning determined to have similar features by an initial machine learning model are classified into the same cluster. For example, some of these clusters perhaps should not be classified into another cluster histopathologically.

The user U can reclassify a plurality of images for learning that have been presented based on their own knowledge. The user U can reclassify each of a plurality of initial clusters to one of a plurality of secondary clusters. In this regard, the plurality of secondary clusters may be, for example, defined by the user U or set by the system 100. Preferably, the user U can define a plurality of secondary clusters based on their own knowledge. Furthermore, it is preferable that a plurality of secondary clusters are determined in accordance with the resolution of a plurality of images for learning. For example, a plurality of secondary clusters for a plurality of images for learning with a lower resolution can be different from a plurality of secondary clusters for a plurality of images for learning with a higher resolution. For example, the user U can determine a plurality of secondary clusters in accordance with the resolution of a plurality of images for learning based on their own knowledge. A plurality of secondary clusters may comprise a cluster for “Others” that do not belong to any classification of interest.

For example, the user U can provide an input regarding which of plurality of secondary clusters each of plurality of images for learning classified into respective plurality of initial clusters displayed on a display of a terminal apparatus can be classified into.

At step S3, an input by the user U is provided to the system 100. The embodiment of providing an input by the user U into the system 100 is not limited. An input by the user U can be inputted into the system 100 in any manner. For example, an input may be inputted from a terminal apparatus into the system 100 through a network (e.g., Internet, LAN, etc.) or, for example, inputted into the system 100 by storing the input in a storage medium with a terminal apparatus and connecting the storage medium to the system 100. The system 100 causes an initial machine learning model to learn information on reclassification by the user U once an input is received. Specifically, the system 100 learns the relationship between a plurality of initial clusters and a plurality of secondary clusters. This can be achieved by, for example, transfer learning of an initial machine learning model.

At step S4, a machine learning model 10 constructed in this manner is provided from the system 100. The machine learning model 10 can classify an inputted image into one of a plurality of secondary clusters. Specifically, classification into a secondary cluster that can be performed based on the knowledge of the user U can be performed by the machine learning model 10. The machine learning model 10 can output a more meaningful classification compared to an initial machine learning model. In this example, the machine learning model 10 can output a classification that is more histopathologically meaningful.

FIG. 1C shows an example of an image of tissue inputted into the machine learning model 10 and an example of a feature map created in accordance with a classification outputted from the machine learning model 10.

FIG. 1C(a) shows an example of an image of tissue inputted into the machine learning model 10. An image of tissue is a WSI of lung tissue of a subject.

FIGS. 1C(b) to 1C(d) show an example of a feature map created in accordance with a classification outputted when a WSI of lung tissue of a subject is inputted into the machine learning model 10. FIG. 1C(b) is a feature map created in accordance with an output from the machine learning model 10 created using an image for learning with a 2× resolution. FIG. 1C(c) is a feature map created in accordance with an output from the machine learning model 10 created using an image for learning with a 5× resolution. FIG. 1C(d) is a feature map created in accordance with an output from the machine learning model 10 created using an image for learning with a 20× resolution.

The feature map of FIG. 1C(b) is separated into four histopathologically meaningful classifications. The feature map of FIG. 1C(c) is separated into eight histopathologically meaningful classifications. The feature map of FIG. 1C(d) is separated into eight histopathologically meaningful classifications. In this manner, classifications vary in accordance with the resolution, and information represented by each feature map varies.

For example, a physician can review such feature maps to diagnose a state related to a disease of a subject. In particular, these feature maps can reflect the knowledge of a specialist or expert, thus enabling a physician without ample experience to render an accurate diagnosis by reviewing the feature maps in which the knowledge of a specialist or expert is reflected.

For example, step S1 to step S4 may be repeated with images classified into a secondary cluster by the machine learning model 10, as a plurality of images for learning, whereby the images classified into the secondary cluster can be subclassified, which can lead to a more detailed diagnosis for the secondary cluster. Images can be further subclassified by repeating the steps.

For example, step S1 to step S4 may be repeated with images classified into the secondary cluster of “Others” by the machine learning model 10, as a plurality of images for learning, whereby the images classified into “Others” can be subclassified. Useful information may be able to be obtained from images deemed to be useless and grouped as “Others”. For example, whether images classified into the secondary cluster of “Others” as corresponding to an artifact are actually an “artifact” can be determined.

FIG. 1D shows a specific example of the flow described above.

A plurality of images for learning are, for example, a plurality of partial images from fragmenting a WSI from tissue staining used in pathological diagnosis into a plurality of regions at a predefined resolution. One partial image is referred to as a tile. In this embodiment, more than 1,000,000 tiles are prepared. At step S1, all of these tiles are inputted into the system 100.

In the system 100, some of the randomly selected tiles (50,000 tiles in this embodiment) among these tiles are extracted, and a machine learning model is created by using these tiles (small set).

For example, an initial machine learning model (Initial Model) is created by self-supervised learning. When a small set is inputted into the created initial machine learning model, features are extracted. An initial cluster is created based on such features (Clustering).

A user (specialist or expert) can reclassify an initial cluster into a secondary cluster, such as Finding A, Finding B, or Others, based on their own knowledge (Integration). A secondary cluster created in this manner is subjected to transfer learning to create a machine learning model (Model).

When all of the tiles are inputted into the created machine learning model (Model), these tiles are classified (Classification) into, for example, Finding A, Finding B, Others, etc. Since a machine learning model (Model) reflects the knowledge of a specialist or expert, an output can be a meaningful classification.

The tiles classified into “Others” can be returned and subjected to the flow described above again, whereby a machine learning that model can subclassify tiles classified into “Others” can be created. Alternatively, tiles classified into “Others” can be returned and inputted into the machine learning model (Model) again, whereby the tiles classified into “Others” can be subclassified.

The flow described above can be materialized by utilizing the system 100 described below.

### 2. Configuration of a System for Creating a Machine Learning Model for Outputting a Feature Map

FIG. 2 shows an example of the configuration of the system 100 for creating a machine learning model for outputting a feature map.

The system 100 is connected to a database unit 200. The system 100 is connected to at least one terminal apparatus 300 via a network 400.

While FIG. 2 shows three terminal apparatuses 300, the number of terminal apparatuses 300 is not limited thereto. Any number of terminal apparatuses 300 can be connected to the system 100 via the network 400.

The network 400 can be any type of network. The network 400 may be, for example, the Internet or LAN. The network 400 may be a wired network or a wireless network.

The system 100 can be, for example, a machine learning model for outputting a feature map or a computer (e.g., server) installed at a service provider providing a feature map. The terminal apparatus 300 may be, for example, a computer (e.g., terminal apparatus) utilized by the user U such as a specialist or expert, or the terminal apparatus 300 may be a computer (e.g., terminal apparatus) utilized by another physician. In this regard, a computer (server or terminal apparatus) can be any type of computer. For example, a terminal apparatus can be any type of terminal apparatus such as a smartphone, tablet, personal computer, smart glass, or smart watch.

The system 100 comprises an interface unit 110, a processing unit 120, and a memory unit 130. The system 100 is connected to the database unit 200.

The interface unit 110 exchanges information with an element external to the system 100. The processing unit 120 of the system 100 can receive information from an element external to the system 100 and transmit information to an element external to the system 100, via the interface unit 110. The interface unit 110 can exchange information in any form. An information terminal used by a first person and an information terminal used by a second person can communicate with the system 100 via the interface unit 110.

The interface unit 110 comprises, for example, an input unit that enables information to be inputted into the system 100. An input unit can enable information to be inputted into the system 100 in any form. If, for example, an input unit is a receiver, information may be inputted by the receiver receiving the information from an element external to the system 100 via a network. In such a case, the network can be of any type. For example, a receiver may receive information via the Internet or LAN.

The interface unit 110 comprises, for example, an output unit that enables information to be outputted from the system 100. An output unit can enable information to be outputted from the system 100 in any form. If, for example, an output unit is a transmitter, information may be outputted by the transmitter transmitting the information to an element external to the system 100 via a network. In such a case, the network can be of any type. For example, a transmitter may transmit information via the Internet or LAN.

The processing unit 120 executes processing of the system 100 and controls the overall operation of the system 100. The processing unit 120 reads out a program stored in a memory unit 150 and executes the program, whereby the system 100 can function as a system for executing desired steps. The processing unit 120 may be implemented by a single processor or a plurality of processors.

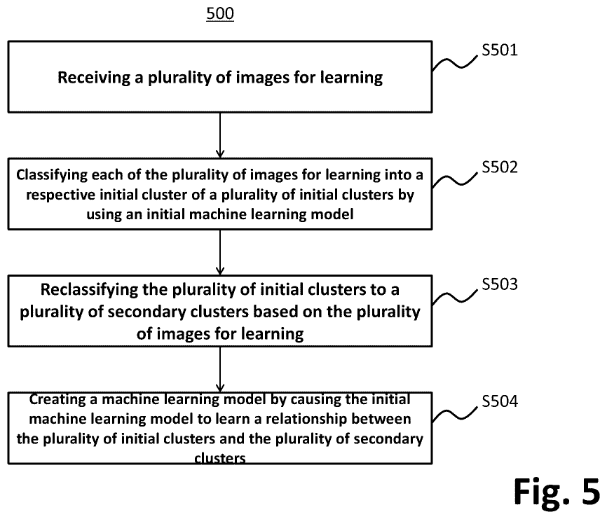

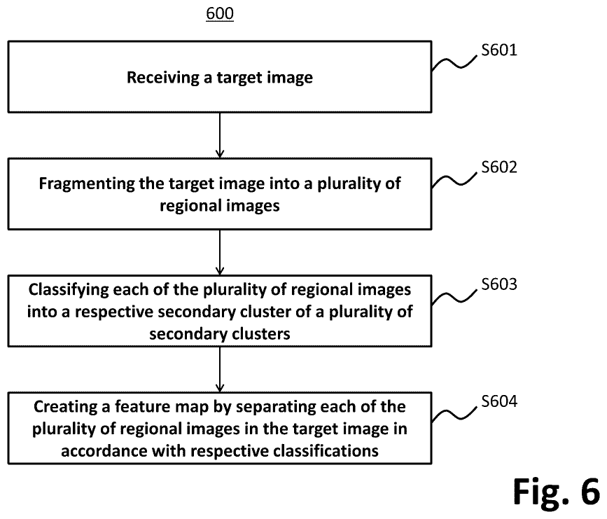

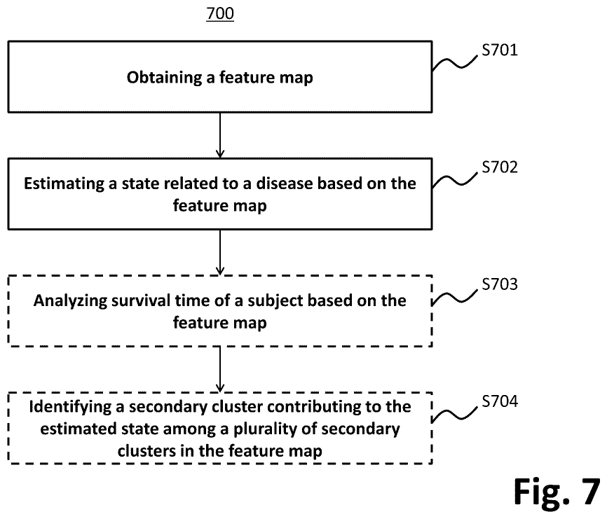

The memory unit 150 stores a program required for the execution of processing of the system 100, data that is required for the execution of the program, etc. The memory unit 150 may store a program for causing the processing unit 120 to perform processing for creating a machine learning model for outputting a feature map (e.g., program for materializing the processing in FIG. 5 described below). The memory unit 150 may store a program for causing the processing unit 120 to perform processing for creating a feature map (e.g., program for materializing the processing in FIG. 6 described below). The memory unit 150 may store a program for causing the processing unit 120 to perform processing for estimating a state related to a disease of a subject (e.g., program for materializing the processing in FIG. 7 described below). In this regard, a program can be stored in the memory unit 150 in any manner. For example, a program may be pre-installed in the memory unit 150. Alternatively, a program may be configured to be installed into the memory unit 150 by download through a network. In such a case, the network can be of any type. The memory unit 150 can be implemented by any storage means. Alternatively, a program may be stored in a machine-readable storage medium and installed into the memory 150 from the storage medium.

For example, a plurality of images for learning can be stored in the database unit 200. A plurality of images for learning can be, for example, data a obtained from a plurality of subjects. For example, the relationship between a plurality of initial clusters and a plurality of secondary clusters can be stored in the database unit 200. For example, the created machine learning model can be stored in the database unit 200. For example, a created feature map can be stored in the database unit 200.

In the example shown in FIG. 2, the database unit 200 is provided external to the system 100, but the present invention is not limited thereto. At least a portion of the database unit 200 can also be provided inside the system 100. At this time, at least a portion of the database unit 200 may be implemented by the same storage means as, or different storage means from, storage means implementing the memory unit 150. In either case, at least a portion of the database unit 200 is configured as a storage section for the system 100. The configuration of the database unit 200 is not limited to a specific hardware configuration. For example, the database unit 200 may be comprised of a single hardware part or a plurality of hardware parts. For example, the database unit 200 may be configured as an external hard disk apparatus of the system 100, or as storage on the cloud connected via the network 400.

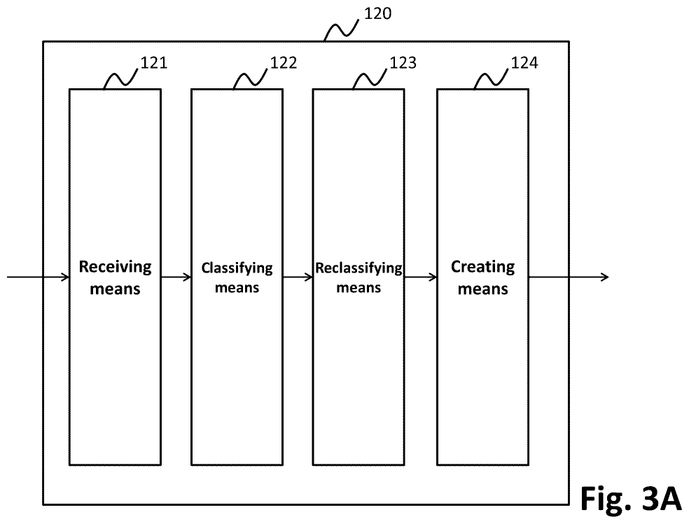

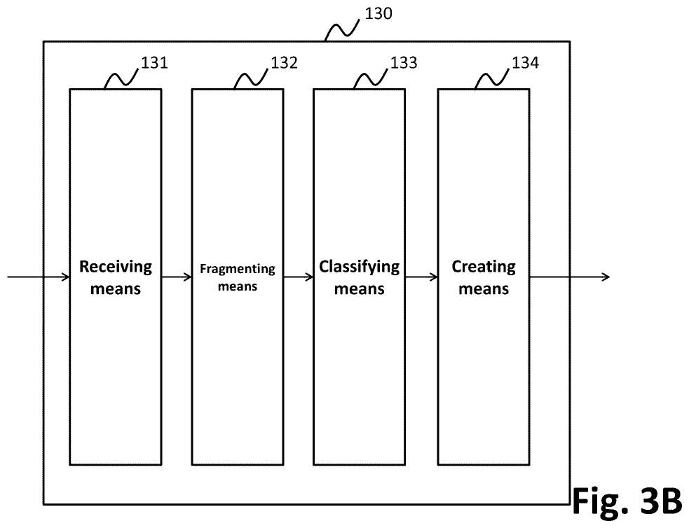

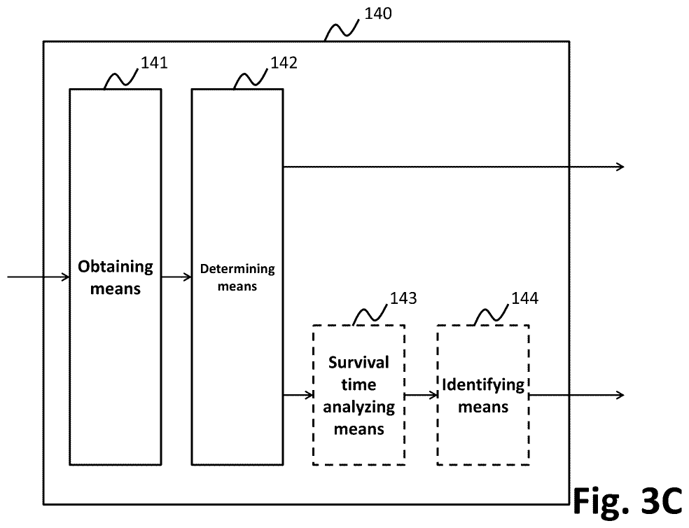

FIG. 3A shows an example of the configuration of the processing unit 120 in one embodiment. The processing unit 120 can have a configuration for the processing for creating a machine learning model for outputting a feature map.

The processing unit 120 comprises receiving means 121, classifying means 122, reclassifying means 123, and creating means 124.

The receiving means 121 is configured to receive a plurality of images for learning. For example, the receiving means 121 can receive a plurality of images for learning received from an element external to the system 100 via the interface unit 110. The receiving means 121 may be configured to, for example, receive a plurality of images for learning from the terminal apparatus 300 via the interface unit 110, receive a plurality of images for learning from the database unit 200 via the interface unit 110, or receive a plurality of images for learning from another source via the interface unit 110. The receiving means 121 can, for example, receive at least some of images classified in accordance with an output from a machine learning model created by the processing unit 120 as a plurality of images for learning.

A plurality of images for learning can be any image in accordance with the application of the created machine learning model. For example, a plurality of images for learning can be images for pathological diagnosis in order to create a machine learning model for creating a feature map that is histopathologically useful. More specifically, a plurality of images for learning can be a plurality of partial images from fragmenting a WSI from tissue staining into a plurality of regions at a predefined resolution. For example, a plurality of images for learning can be a plurality of partial images from fragmenting a radiographic image into a plurality of regions at a predefined resolution in order to create a machine learning model for creating a feature map that is useful for X-ray diagnosis. A predefined resolution can be any resolution, such as about 2× resolution, about 5× resolution, about 10× resolution, about 15× resolution, or about 20× resolution. For example, images of a plurality of subjects having various diseases can be used as a plurality of images for learning in order to create a machine learning model that is capable of outputting classifications for various diseases. Specifically, images of various cancer cells can be used as a plurality of images for learning in order to create a machine learning model that is capable of outputting classifications for various cancers.

Data used for learning in the present invention does not need to be necessarily image data. A machine learning model can be created by using data other than image data instead of images for learning in learning of the invention.

In one example, a plurality of images for learning can comprise an image of tissue of a subject with interstitial pneumonia and an image of tissue of a subject without interstitial pneumonia in order to create a machine learning model for creating a feature map which enables estimation of the presence/absence of interstitial pneumonia. At this time, an image of tissue can be a plurality of partial images from fragmentation into a plurality of regions at a predefined resolution.

A plurality of images for learning are passed onto the classifying means 122.

The classifying means 122 is configured to classify each plurality of images for learning into a respective initial cluster of a plurality of initial clusters. The classifying means 122 can classify each of a plurality of images for learning into a respective initial cluster of a plurality of initial clusters by using an initial machine learning model.

An initial machine learning model is any machine learning model caused to at least learn to output a feature of an inputted image from the image. An initial machine learning model can be, for example, a machine learning model based on convolutional neutral network (CNN). More specifically, CNN can be, for example, ResNet18.

An initial machine learning model can be constructed by any approach. An initial machine learning model may be constructed by, for example, supervised learning or unsupervised learning. Preferably, an initial machine learning model can be constructed by Self-Supervised Learning. In one example, a CNN-based machine learning model is caused to learn a plurality of images for initial learning by self-supervised learning. A plurality of images for initial learning may be the same or similar images as a plurality of images for learning. Each of a plurality of images for learning do not need to be labeled by using self-supervised learning. An initial machine learning model that has learned in this manner would output a feature of an inputted image from the image.

For example, the classifying means 122 can classify a feature outputted from an initial machine learning model to one of a plurality of initial clusters by using a clustering model. A clustering model is caused to learn to cluster an inputted feature by any clustering approach. For example, a clustering model can cluster an inputted feature by k-means clustering.

A plurality of initial clusters can comprise any number of initial clusters. For example, a plurality of initial clusters can comprise 5, 8, 10, 30, 50, 80, 100, 120 initial clusters, etc. If the number of initial clusters is too low, the possibility of images for learning with different significance being classified into the same initial cluster would be high. If the number of initial clusters is too high, the possibility of images for learning having the same significance being classified into different initial clusters would be high. It is preferable to set a suitable number of initial clusters in accordance with the details of the images for learning.

If an initial machine learning model is connected to a clustering model in this manner, an image inputted into the initial machine learning model would be classified into one of a plurality of initial clusters.

The aforementioned example describes that the initial machine learning model and clustering model are separate models, but the present invention is not limited thereto. For example, an initial machine learning model may be constructed to directly classify an inputted image into one of a plurality of initial clusters, i.e., constructed so that a clustering model is integrated into an initial machine learning model.

The reclassifying means 123 is configured to reclassify a plurality of initial clusters into a plurality of secondary clusters based on a plurality of: images for learning classified into respective plurality of initial clusters. The reclassifying means 123 may be configured to, for example, automatically reclassify based on a plurality of images for learning classified into respective plurality of initial clusters or reclassify in accordance with an external input. In this regard, a plurality of secondary clusters may be, for example, defined by a user, preset, or dynamically varied. Preferably, users can define a plurality of secondary clusters based on their own knowledge. Furthermore, it is preferable that a plurality of secondary clusters are determined in accordance with the resolution of a plurality of images for learning. For example, a plurality of secondary clusters for a plurality of images for learning with a lower resolution can be different from a plurality of secondary clusters for a plurality of images for learning with a higher resolution. For example, users can determine a plurality of secondary clusters in accordance with the resolution of a plurality of images for learning based on their own knowledge.

When reclassifying in accordance with an external input, the reclassifying means 123 can reclassify, for example, in accordance with an input from a user. It is preferable that a user is, for example, a specialist or expert because this enables knowledge of the specialist or expert to be integrated into classification. For example, for pathological diagnosis, secondary clusters with a pathological meaning may be defined based on the user's own knowledge and each of the initial clusters (all or part of the initial clusters) may be classified into the secondary clusters.

For example, the reclassifying means 123 can present a plurality of images for learning, which have been classified into respective plurality of initial clusters by the classifying means 122, to a user. For example, a plurality of images for learning can be presented to a user by outputting the images out of the system 100 via the interface unit 110. For example, a plurality of images for learning can be displayed on a display unit of the terminal apparatus 300 in a manner shown in FIG. 1B. A user can view the images and associate each of a plurality of initial clusters to one of a plurality 41 secondary clusters. When a user inputs the associated user input into the terminal apparatus 300, the reclassifying means 123 can receive the associated user input via the user interface 110. In addition, the reclassifying means 123 can reclassify a plurality of initial clusters into a plurality of secondary clusters based on the associated user input.

When automatically reclassifying, the reclassifying means 123 may, for example, reclassify a plurality of initial clusters into a plurality of secondary clusters in a rule-based manner, or reclassify a plurality of initial clusters into a plurality of secondary clusters by utilizing another machine learning model.

The creating means 124 is configured to create a machine learning model by causing an initial machine learning model to learn the relationship between a plurality of initial clusters and a plurality of secondary clusters. An initial machine learning model can be caused to learn the relationship between a plurality of initial clusters and a plurality of secondary clusters by using an approach that is known, or will be known in the future, in the art. The creating means 124 can create a machine learning model by, for example, subjecting an initial machine learning model to transfer learning by using the relationship between a plurality of initial clusters and a plurality of secondary clusters.

In one example, the creating means 124 can add a fully connected (FC) layer to a CNN-based initial machine learning model and optimize the weighting of the FC layer to cause the initial machine learning model to learn the relationship between a plurality of initial clusters and a plurality of secondary clusters. At this time, not only the weighting of the FC layer, but also parameters of at least one layer of the CNN may be configured to be adjusted.

When an image is inputted, a machine learning model created in this manner can classify the image into one of a plurality of secondary clusters. Even if a classification is not meaningful in an initial cluster, a meaningful classification can be outputted by classifying an image into a secondary cluster.

For example, if an image is fragmented into a plurality of regional images and the plurality of regional images are inputted into such a machine learning model, each of the plurality of regional images would be classified into one of a plurality of secondary clusters. A feature map can be created by separating each of the plurality of regional images in one image in accordance with respective classifications.

If, for example, a plurality of secondary clusters are configured to represent respective diseases by using images of a plurality of subjects having various diseases as a plurality of images for learning, the created machine learning model would output which disease cluster the disease indicated by an inputted image is classified into.

If, for example, an image obtained from a subject with an unknown disease is inputted into such a machine learning model, the image would be classified into one of plurality of secondary clusters representing a plurality of diseases. Specifically, looking at which secondary cluster the image is classified into enables what disease the subject has to be known. As a more specific example, if an image obtained from a subject having some type of cancer is inputted into the machine learning model, the image would be classified into one of a plurality of secondary clusters representing various cancers. A physician can diagnose whether the cancer the subject has is lung cancer, gastric cancer, liver cancer, etc. from this classification.

For example, a machine learning model created by the processing unit 120 is outputted out from the system 100 via the interface unit 110. For example, a machine learning model may be transmitted to the database unit 200 via the interface unit 110 and stored in the database unit 200. Alternatively, a machine learning model may be transmitted to the processing unit 130 described below for creating a feature map. As described below, the processing unit 130 may be a constituent element of the same system 100 as the processing unit 120 or a constituent element of another system.

FIG. 3B shows an example of the configuration of the processing unit 130 in another embodiment. The processing unit 130 can have a configuration for the processing to create a feature map. The processing unit 130 may be a processing unit the system 100 comprises in place of the processing unit 120 described above, or a processing unit the system 100 comprises in addition to the processing unit 120. If the processing unit 130 is a processing unit the system 100 comprises in addition to the processing unit 120, the processing unit 120 and the processing unit 130 may be implemented by the same processor or different processors.

The processing unit 130 comprises receiving means 131, fragmenting means 132, classifying means 133, and creating means 134.

The receiving means 131 is configured to receive a target image. A target image is an image of a target for which a feature map is created. A target image can be, for example, any image obtained from the body of a subject (e.g., WSI from tissue staining, radiographic image (e.g., tomographic image such as CT), etc.). For example, the receiving means 131 can receive a target image received from an element external to the system 100 via the interface unit 110. The receiving means 131 may be configured to, for example, receive a target image from the terminal apparatus 300 via the interface unit 110, receive a target image from the database unit 200 via the interface unit 110, or receive a target image from another source via the interface unit 110.

The fragmenting means 132 is configured to fragment a target image into a plurality of regional images. The fragmenting means 132 can fragment a target image into a plurality of regional images at a predefined resolution. The predefined resolution can be, for example, about 2× resolution, about 5× resolution, about 10× resolution, about 15× resolution, about 20× resolution, etc. A suitable resolution can be chosen in accordance with the objective of a feature map. The fragmenting means 132 can fragment a target image into a plurality of regional images by using an approach that is known, or will be known in the future, in the field of image processing.

The classifying means 133 is configured to classify each of a plurality of regional images into a respective secondary cluster of a plurality of secondary clusters. The classifying means 133 can classify each of a plurality of regional images into respective secondary clusters by inputting the plurality of regional images into a machine learning model. In this regard, the machine learning model may be a machine learning model created by the processing unit 120 described above or a machine learning model created in another manner, as long as an inputted image can be classified into one of a plurality of secondary clusters.

For example, if the first regional image of a plurality of regional images is inputted into a machine learning model, the first regional image is classified into a corresponding secondary cluster, if the second regional image of the plurality of regional images is inputted into the machine learning model, the second regional image is classified into a corresponding secondary cluster . . . if the nth regional image of the plurality of regional images is inputted into the machine learning model, the nth regional image is classified into the corresponding secondary cluster.

The creating means 134 is configured to create a feature map by separating each of a plurality of regional images in a target image in accordance with respective classifications. The creating means 134 can create a feature map by, for example, regional images belong to the same classification among a plurality of regional images with the same color. For example, the feature maps shown in FIGS. 1C(b) to 1C(d) can be created by the creating means 134.

With such a feature map, it is possible to visually understand what each of a plurality of regions within a target image is like. Even information that cannot be visually understood from a target image can be visually understood with a feature map. This is useful particularly in, for example, pathological diagnosis, etc.

For example, a feature map created by the processing unit 130 is outputted out of the system 100 via the interface unit 110. For example, a feature map may be transmitted to the database unit 200 via the interface unit 110 and stored in the database unit 200. Alternatively, a feature map may be transmitted to a processing unit 140 described below for the processing to estimate a state related to a disease of a subject. As described below, the processing unit 140 may be a constituent element of the same system 100 as the processing unit 130 or a constituent element of another system.

FIG. 3C shows an example of the configuration of the processing unit 140 in yet another embodiment. The unit 140 can have a configuration for the processing processing to estimate a state related to a disease of a subject. The processing unit 140 may be a processing unit that the system 100 comprises in place of the processing unit 120 and processing unit 130 described above, or a processing unit that the system 100 comprises in addition to the processing unit 120 and/or processing unit 130 described above. If the processing unit 140 is a processing unit that the system 100 comprises in addition to the processing unit 120 and/or processing unit 130, all of the processing unit 120, processing unit 130, and processing unit 140 may be implemented by the same processor, all of them may be implemented by different processors, or two of the processing unit 120, processing unit 130, and processing unit 140 may be implemented by the same processor.

The processing unit 140 comprises obtaining means 141 and estimating means 142.

The obtaining means 141 is configured to obtain a feature map. In this regard, the obtained feature map may be a feature map created by the processing unit 130 described above or a feature map created in another manner, as long as the feature map is created from an image of tissue of a subject. For example, a machine learning model used in creating a feature map may be a machine learning model created by the processing unit 120 described above or a machine learning model created in another manner, as long as an inputted image can be classified into one of a plurality of secondary clusters.

For example, the obtaining means 141 may be configured to obtain a plurality of feature maps. For example, a plurality of feature maps can be a plurality of feature maps created from a plurality of images of tissue obtained from different tissues. For example, a plurality of feature maps can be a plurality of feature maps created from a plurality of images of tissue of different types. For example, a plurality of feature maps can be a plurality of feature maps created at different resolutions from the same image of tissue. Precision of estimation by the subsequent estimating means 143 can be enhanced by utilizing a plurality of feature maps.

The estimating means 142 is configured to estimate a state related to a disease of a subject based on a feature map. The estimating means 142 can estimate, for example, whether a subject has some type of a disease, whether a subject has a specific disease (e.g., interstitial pneumonia (IP) or usual interstitial pneumonia (UIP)), or what type of disease the specific disease of a subject is (e.g., which type of interstitial pneumonia), based on a feature map. For example, whether a subject has interstitial pneumonia (IP), usual interstitial pneumonia (UIP), or the type of interstitial pneumonia of a subject, can be estimated based on a feature map created from an image of tissue obtained from a lung of the subject.

For example, the estimating means 142 can estimate a state related to a disease of a subject based on information extracted from a feature map. For example, the estimating means 142 can calculate the frequency of each of a plurality of secondary clusters from a feature map and estimate a state related to a disease based on the calculated frequency. The frequency of each of a plurality of secondary clusters can be calculated by counting the number of image regions belonging to a secondary cluster for each of a plurality of secondary clusters and normalizing with the total number of image regions. For example, the estimating means 142 can estimate a state related to a disease of a subject from a secondary cluster with a high frequency. The estimating means 142 can utilize not only the frequency described above, but also any other information extracted from a feature map. The estimating means 142 can utilize, for example, positional information of each secondary cluster in a feature map. The estimating means 142 can estimate a state related to a disease of a subject by using any approach that is known, or will be known in the future, in the technical field. For example, the estimating means 142 can classify and estimate a state related to a disease of a subject by using a classifier such as random forest or support vector machine.

For example, the estimating means 142 can estimate a state related to a disease of a subject by utilizing a machine learning model for estimation caused to learn the relationship between a feature map and the state related to a disease of a subject. A machine learning model for estimation can be a machine learning model based on a neural network (e.g., CNN) that enables estimation based on an image. For example, a machine learning model for estimation can be constructed by causing learning using a feature map of a subject as input supervisor data and a state related to disease of the subject as output supervisor data. If a feature map subject is inputted into a machine learning model for estimation constructed in this manner, a state related to a disease of the subject is outputted.

When the obtaining means 141 has obtained a plurality of feature maps, the estimating means 142 can estimate a state related to a disease of a subject based on the plurality of feature maps.

For example, the estimating means 142 can estimate a state related to a disease of a subject based on information extracted from a plurality of feature maps. For example, the estimating means 142 can calculate the frequency of each of a plurality of secondary clusters from each of the plurality of feature maps and estimate a state related to a disease based on the calculated frequency. The frequency of each of a plurality of secondary clusters can be calculated by counting the number of image regions belonging to a secondary cluster over a plurality of feature maps for each of the plurality of secondary clusters and normalizing with the total number of image regions. For example, the estimating means 142 can estimate a state related to a disease of a subject from a secondary cluster with a high frequency. The estimating means 142 can utilize not only the frequency described above, but also any other information extracted from a plurality of feature maps.

For example, the estimating means 142 can estimate a state related to a disease of a subject by utilizing a machine learning model for estimation caused to learn the relationship between a feature map and the state related to a disease of a subject. A machine learning model for estimation can be a machine learning model based on a neural network (e.g., CNN) that enables estimation based on an image. For example, a machine learning model for estimation can be constructed by causing learning using a feature map of a subject as input supervisor data and a state related to a disease of the subject as output supervisor data. If a plurality of feature maps of a new subject are inputted into a machine learning model for estimation constructed in this manner, a state related to a disease of the subject is outputted for each feature map. A state related to a disease of the subject can be estimated based on the plurality of outputs.

For example, the estimating means 142 can, by using a plurality of feature maps, identify an error in at least one of a plurality of feature maps and estimate a state related to a disease of a subject based on at least one feature excluding the at least one feature map in which an error has been identified. For example, if a secondary cluster to which a certain region is classified is clearly inconsistent with a secondary cluster to which a region corresponding to another feature map is classified in a first feature map among a plurality of feature maps, the first feature map can be deemed as likely to have an error. In such a case, the estimating means 142 can estimate a state related to a disease of a subject without using the first map. Since a feature map that is likely to include an error is excluded for estimation, the precision of estimation can be enhanced.

In one example, the estimating means 142 can estimate the type of interstitial pneumonia, e.g., whether interstitial pneumonia is usual interstitial pneumonia, based on a feature map created from an image of tissue of a lung of a subject having interstitial pneumonia. In this example, the estimating means 142 can estimate the type of interstitial pneumonia of a subject, e.g., classify whether interstitial pneumonia is usual interstitial pneumonia, by calculating the frequency for each of a plurality of secondary clusters contained in the feature map created from the image of tissue of a lung of the subject and performing random forest on the calculated frequency.

The processing unit 140 can further analyze whether a classification of a plurality of secondary clusters contributed to a state estimated by the estimating means 142. For this reason, the processing unit 140 can further comprise survival time analyzing means 143 and identifying means 144.

The survival time analyzing means 143 is configured to analyze the survival time of a subject based on a feature map. The survival time analyzing means 143 can analyze the survival time by using any approach that is known, or will be known in the future, in the technical field. The survival time analyzing means 143 can analyze the survival time of a subject by using, for example, Kaplan-Meier method, log-rank test, Cox proportional hazards model, etc.

The identifying means 144 is configured to identify at least one secondary cluster contributing to an estimated state of a subject among a plurality of secondary clusters in a feature map from a result of analyzing the survival time by the survival time analyzing means 143. The identifying means 144 can, for example, identify a secondary cluster with a high hazard ratio obtained by survival time analysis as a secondary cluster contributing to the estimated state. A secondary cluster with a high hazard ratio can be, for example, a secondary cluster having the highest hazard ratio, a secondary cluster having a hazard ratio equal to or greater than a predefined threshold value, etc.

A factor unique to a subject having a disease with poor prognosis such as usual interstitial pneumonia can be identified by analyzing what the factor contributing to the estimated state is in this manner. Such a factor can be utilized as an indicator in diagnosis. This can lead to accurate and simple diagnosis.

For example, a state related to a disease of a subject estimated by the processing unit 140 is outputted out of the system 100 via the interface unit 110. For example, an output can be transmitted to the terminal apparatus 300 via the interface unit 110, whereby a physician utilizing the terminal apparatus 300 can utilize the output as an indicator for diagnosis.

The aforementioned example has described estimation of a state related to a disease of a subject by the processing unit 140, but the target of estimation by the processing unit 140 is not limited thereto. Anything can be estimated in accordance with a feature represented by a feature map.

Each constituent element of the system 100 described above may be comprised of a single hardware part or a plurality of hardware parts. If comprised of a plurality of hardware parts, each hardware part may be connected in any manner. Each hardware part may be connected via a wireless connection or a wired connection. The system 100 of the invention is not limited to a specific hardware configuration. The processing unit 120, processing unit 130, and processing unit 140 comprised of an analog circuit instead of a digital circuit are also within the scope of the invention. The configuration of the system 100 of the invention is not limited to those described above, as long as the function thereof can be materialized.

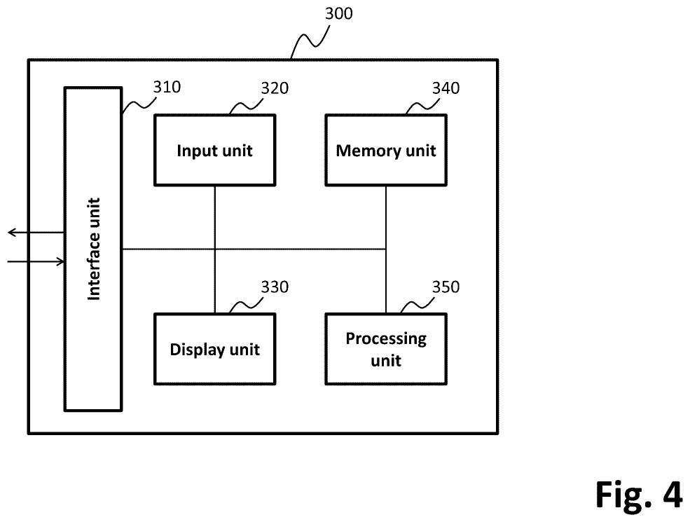

FIG. 4 shows an example of the configuration of the terminal apparatus 300.

The terminal apparatus 300 comprises an interface unit 310, an input unit 320, a display unit 330, a memory unit 340, and a processing unit 350.

The interface unit 310 controls communication via the network 400. The processing unit 350 of the terminal apparatus 300 can receive information from an element external to the terminal apparatus 300 and transmit information to an element external to the terminal apparatus 300 via the interface unit 310. The interface unit 310 can control communication by any method.

The input unit 320 enables a user to input information into the terminal apparatus 300. The input unit 320 can enable a user to input information into the terminal apparatus 300 in any manner. If, for example, the input unit 320 is a touch panel, information may be inputted by a user touching the touch panel. Alternatively, if the input unit 320 is a mouse, the input unit may be configured so that a user inputs information by operating the mouse. Alternatively, if the input unit 320 is a keyboard, the input unit may be configured so that a user inputs information by pressing a key on the keyboard. Alternatively, if the input unit is a microphone, the input unit may be configured so that a user inputs information by inputting a voice into the microphone. Alternatively, if the input unit is a data reader, the input unit may be configured so that information is inputted by reading out information from a storage medium connected to the computer system 100.

The display unit 330 can be any display for displaying information. For example, the display unit 330 can display an image of an initial cluster as shown in FIG. 1B.

The memory unit 340 stores a program for the execution of processing in the terminal apparatus 300, data that is required for the execution of the program, etc. The memory unit 340 may store an application implementing any function. In this regard, a program can be stored in the memory unit 340 in any manner. For example, a program may be preinstalled in the memory unit 340. Alternatively, a program may be configured to be installed into the memory unit 340 by download through the network 400. The memory unit 340 can be implemented by any storage means.

The processing unit 350 controls the overall operation of the terminal apparatus 300. The processing unit 350 reads out a program stored in the memory unit 340 and executes the program, whereby the terminal apparatus 300 can function as an apparatus for executing desired steps. The processing unit 350 may be implemented by a single processor or a plurality of processors.

In the example shown in FIG. 4, each constituent element of the terminal apparatus 300 is provided within the terminal apparatus 300, but the present invention is not limited thereto. Any of the constituent elements of the terminal apparatus 300 can be provided external to the terminal apparatus 300. If, for example, each of the input unit 320, display unit 330, memory unit 340, and processing unit 350 is comprised of separate hardware parts, each hardware member may be connected via any network. At this time, the network may be of any type. Each hardware part may be connected, for example, via a LAN, a wireless connection, or a wired connection. The terminal apparatus 300 is not limited to a specific hardware configuration. For example, the processing unit 350 comprised of an analog circuit instead of a digital circuit is also within the scope of the invention. The configuration of the terminal apparatus 300 is not limited to those described above, as long as the function thereof can be materialized.

### 3. Processing in a System for Creating a Machine Learning Model for Outputting a Feature Map

FIG. 5 shows an example of processing in the system 100. Processing 500 is processing for creating a machine learning model for outputting a feature map. The processing 500 is executed in the processing unit 120 of the system 100.

At step S501, the receiving means 121 of the processing unit 120 receives a plurality of images for learning. For example, the receiving means 121 can receive a plurality of images for learning received from an element external to the system 100 via the interface unit 110. The receiving means 121 may, for example, receive a plurality of images for learning from the terminal apparatus 300 via the interface unit 110, receive a plurality of images for learning from the database unit 200 via the interface unit 110, or receive a plurality of images for learning from another source via the interface unit 110. For example, the receiving means 121 can receive some (e.g., images for learning reclassified into the secondary cluster of “Others”) of a plurality of images for learning reclassified into at least one of a plurality of secondary clusters at step S503 described below. For example, the receiving means 121 can receive a plurality of images classified into at least one of a plurality of secondary clusters by a machine learning model created at step S504 described below (e.g., images classified into the secondary cluster of “Others”).

A plurality of images for learning can be any image in accordance with the application of the created machine learning model. For example, a plurality of images for learning can be a plurality of partial images from fragmenting a WSI from tissue staining into a plurality of regions at a predefined resolution in order to create a machine learning model for creating a feature map that is histopathologically useful. For example, a plurality of images for learning can be a plurality of partial images from fragmenting a radiographic image into a plurality of regions at a predefined resolution. The predefined resolution can be any resolution, such as about 2× resolution, about 5× resolution, about 10× resolution, about 15× resolution, or about 20× resolution.

At step S502, classifying means 122 of the processing unit 120 classifies each of the plurality of images for learning received at step S502 into a respective initial cluster of a plurality of initial clusters. The classifying means 122 can classify each of a plurality of images for learning into a respective initial cluster of a plurality of initial clusters by using an initial machine learning model. An initial machine learning model can be any machine learning model caused to at least learn to output a feature of an outputted image from the image. For example, the classifying means 122 may be configured to classify by combining an initial machine learning model and a clustering model for clustering outputs of the initial model into an machine learning initial cluster, or configured to classify by using an initial machine learning model constructed as classifying means directly classifying an inputted image into one of a plurality of initial clusters.

At step S503, the reclassifying means 123 of the processing unit 120 reclassifies a plurality of initial clusters into a plurality of secondary clusters based on the plurality of images for learning classified at step S502. For example, the reclassifying means 123 may be configured to automatically reclassify based on a plurality of images for learning classified into respective plurality of initial clusters or reclassify in accordance with an external input.

When reclassifying in accordance with an external input, step S503 can comprise steps of presenting a plurality of images for learning classified at step S502 to a user (e.g., specialist or expert), receiving a input for user associating each of a plurality of initial clusters with one of a plurality of secondary clusters, and reclassifying the plurality of initial clusters into the plurality of secondary clusters based on the user input, by the reclassifying means 123. For example, at the presenting step, the reclassifying means 123 can present a plurality of images for learning to a user by outputting a plurality of images for learning out of the system 100 via the interface unit 110. For example, a plurality of images for learning can be displayed on a display unit of the terminal apparatus 300 in a manner shown in FIG. 1B. A user can view the images and input a user input associating each of a plurality of initial clusters to one of a plurality of secondary clusters into the terminal apparatus 300. At the step of receiving a user input, the reclassifying means 123 can receive a user input via the user interface 110.

When automatically reclassifying, the reclassifying means 123 may, for example, reclassify a plurality of initial clusters into a plurality of secondary clusters in a rule-based manner, or reclassify a plurality of initial clusters into a plurality of secondary clusters by utilizing another machine learning model.

At step S504, the creating means 124 of the processing unit 120 creates a machine learning model by causing an initial machine learning model to learn the relationship between a plurality of initial clusters and a plurality of secondary clusters. The creating means 124 can create a machine learning model by, for example, subjecting an initial machine learning model to transfer learning using the relationship between a plurality of initial clusters and a plurality of secondary clusters.

A machine learning model for outputting a feature map is created by the processing 500 described above. When an image is inputted, a machine learning model created in this manner can classify the image into one of a plurality of secondary clusters. Even if a classification is not meaningful in an initial cluster, a meaningful classification can be outputted by classifying an image into a secondary cluster, whereby a feature model with a meaningful classification can be created and outputted. A created machine learning model can be utilized in processing 600 and processing 700 described below.

For example, prior to step S504, steps S501 to step S503 may be repeated by using some (e.g., images for learning reclassified into the secondary cluster of “Others”) of a plurality of images for learning reclassified into at least one of a plurality of secondary clusters at step S503, whereby a machine learning model capable of subclassifying an image classified into the secondary cluster can be created at step S504. For example, an image classified into the secondary cluster of “Others” can be deemed not useful, or as an “artifact” or “noise”. However, it is possible to create a machine learning model which can classify an image that is truly not useful and other images by repeating step S501 to step S503 from using images classified into the secondary cluster of “Others”. For example, an image showing ink and an image without ink can be more accurately classified by repeating steps S501 to Step S503 on images classified into a secondary cluster as an image showing ink used for marking in the image.

This enables useful information to be obtained from an image buried as a secondary cluster of “Others”. Alternatively, information that is not an “artifact” or “noise” can be extracted from an image deemed as an “artifact” or “noise”.

This can be achieved, for example, by repeating the processing 500 by using an image classified into at least one secondary cluster (e.g., image classified into the secondary cluster of “Others”) among a plurality of secondary clusters by a machine learning model created through the processing 500. It should be noted that such an embodiment can contain noise in an output from a machine learning model.

For example, an image classified into at least one secondary cluster (e.g., image classified into the secondary cluster of “Others”) among a plurality of secondary clusters by a machine learning model created through the processing 500 can be inputted into the machine learning model again to subclassify the image classified into the secondary cluster.

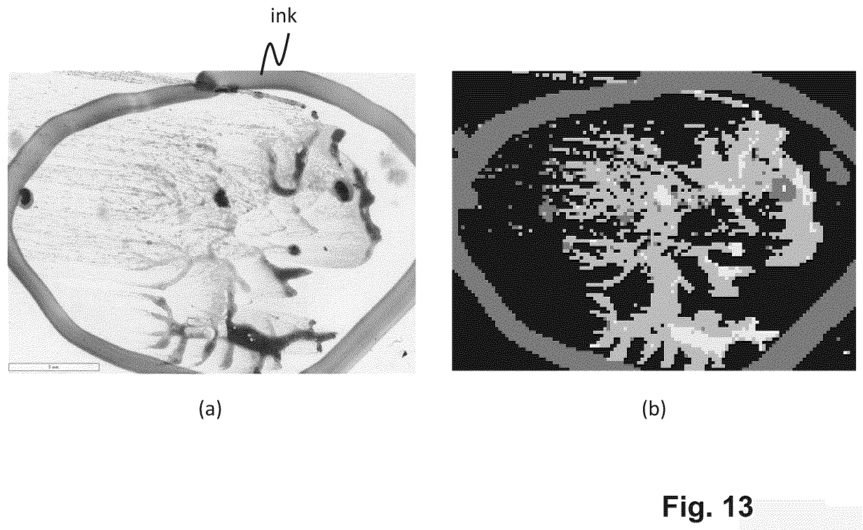

FIG. 13 shows image (a) of a cell marked with ink and a feature map created from the image.

This example repeated processing of inputting a partial image classified as an “artifact” by a machine learning model into the machine learning model again, and inputting the partial image classified into “artifact”, which is an output thereof, into the machine learning model again.

It can be understood from FIG. 13 that the portion of an artifact is clearly separated. If the portion of an artifact can be clearly separated in this manner, the precision of classification of portions other than the artifact, i.e., portion of interest, can be enhanced.

While the aforementioned example describes that a machine learning model for outputting a feature map is created, the application of the created machine learning model is not limited to outputting a feature map. For example, a machine learning model can be used for determining the type of a disease of a subject. For example, a physician can diagnose a disease of a subject by using an output from a machine learning model as an indicator.

When creating a machine learning model for determining the type of a disease of a subject, the receiving means 121 of the processing unit 120 receives images of a plurality of subjects having various diseases as a plurality of images for learning at step S501. For example, a plurality of images for learning can comprise an image obtained from a subject having lung cancer, an image obtained from a subject having gastric cancer, an image obtained from a subject having liver cancer . . . . An image may be, for example, a WSI from tissue staining, high resolution tomographic image, or projectional X-ray image of the chest.

At step S502, the classifying means 122 of the processing unit 120 classifies each of a plurality of images for learning received at step S502 to a respective initial cluster of a plurality of initial clusters.

At step S503, the reclassifying means 123 of the processing unit 120 reclassifies a plurality of initial clusters into a plurality of secondary clusters based on the plurality of images for learning classified at step S502. For example, the reclassifying means 123 may automatically reclassify based on a plurality of images for learning classified into the respective plurality of initial clusters or reclassify in accordance with an external input. The reclassifying means 123 can reclassify initial clusters into a plurality of secondary clusters each corresponding to a single disease. For example, secondary clusters correspond to respective cancer, e.g., the first secondary cluster corresponds to lung cancer, the second secondary cluster corresponds to gastric cancer, the third secondary cluster corresponds to liver cancer, etc. For example, this can be performed by a user (e.g., specialist or expert) viewing each image and inputting a user input associating each of a plurality of initial clusters to one of a plurality of secondary clusters into the terminal apparatus 300.

At step S504, the creating means 124 of the processing unit 120 creates a machine learning model by causing an initial machine learning model to learn the relationship between a plurality of initial clusters and a plurality of secondary clusters.

If an image obtained from a subject with an unknown disease is inputted into a machine learning model created in this manner, which secondary cluster the image is classified into is outputted, whereby a physician can determine that a disease corresponding to the secondary cluster is the disease the subject has.

FIG. 6 shows another example of processing in the system 100. The processing 600 is processing for creating a feature map. The processing 600 is executed in the processing unit 130 of the system 100.

At step S601, the receiving means 131 of the processing 130 receives a target image. For example, the receiving means 131 can receive a target image received from an element external to the system 100 via the interface unit 110. The receiving means 131 may, for example, receive a target image from the terminal apparatus 300 via the interface unit 110, receive a target image from the database unit 200 via the interface unit 110, or receive a target image from another source via the interface unit 110.

A target image is an image of a target for which a feature map is created. A target image can be, for example, any image obtained from the body of a subject (e.g., WSI from tissue staining, radiographic image, etc. of tissue).

At step S602, fragmenting means 132 of the processing unit 130 fragments the target image received at step S601 into a plurality of regional images. The fragmenting means 132 can fragment a target image into a plurality of regional images at a predefined resolution. The predefined resolutions can be, for example, about 2× resolution, about 5× resolution, about 10× resolution, about 15× resolution, about 20× resolution, etc. A target image can be fragmented at a suitable resolution in accordance with the objective of the created feature map.

At step S603, the classifying means 133 of the processing unit 130 classifies each of a plurality of regional images fragmented at step S602 into a respective secondary cluster of a plurality of secondary clusters. The classifying means 133 can classify a plurality of regional images into respective secondary clusters by inputting the plurality of regional images into a machine learning model.

A machine learning model may be a machine learning model created by the processing 500 or a machine learning model created in another manner. Since a secondary cluster can be a classification reflecting knowledge of a specialist of expert, classification by the classification means 133 can be classification integrating the knowledge of a specialist of expert.

At step S604, the creating means 134 of the processing unit 130 creates a feature map by separating each of a plurality of regional images in a target image in accordance with respective classifications. At step S604, the creating means 134 can create a feature map by, for example, coloring regional images belonging to the same classification among a plurality of regional images with the same color.

Such a feature map enables visually understanding what kind of region each of a plurality of regions in a target image is. Even information that cannot be visually understood from a target image can be visually understood with a feature map. Since separation in a feature map can be in accordance with a classification reflecting the knowledge of a specialist or expert, the feature map can integrate the knowledge of a specialist or expert.

A feature map is created through the processing 600 described above. A feature map created in this manner can be utilized in processing 700 described below.

FIG. 7 shows another example of processing in the system 100. The processing 700 is processing for estimating a state related to a disease of a subject. The processing 700 is executed in the processing unit 140 of the system 100.

At step S701, the obtaining means 141 of the processing unit 140 obtains a feature map. A feature map is a feature map created from an image of tissue of a subject. A feature map may be a feature map created through the processing 600, or a feature map created in another manner.

For example, the obtaining means 141 may be configured to obtain a plurality of feature maps.

At step S702, the estimating means 142 of the processing unit 140 estimates a state related to a disease of a subject based on a feature map. At step S702, the estimating means 142 can estimate, for example, whether a subject has some type of a disease, whether a subject has a specific disease (e.g., interstitial pneumonia (IP) or usual interstitial pneumonia (UIP)), what type of disease the specific disease of a subject is (e.g., which type of interstitial pneumonia), or the severity of a specific disease of a subject (e.g., severity of one of interstitial pneumonia). For example, whether a subject has interstitial pneumonia (IP), usual interstitial pneumonia (UIP), type of interstitial pneumonia of a subject, or severity of interstitial pneumonia of a subject can be estimated based on a feature map created from an image of tissue obtained from a lung of a subject.

The estimating means 142 can estimate a state related to a disease of a subject based on information extracted from a feature map. Information extracted from a feature map may be, for example, frequency of each of a plurality of secondary clusters, positional information of each secondary cluster in a feature map, or image of a feature map itself.

If a plurality of feature maps are obtained at step S701, the estimating means 142 can estimate a state related to a disease of a subject based on the plurality of feature maps at step S702.

For example, the estimating means 142 may be configured to estimate a state related to a disease of a subject based on information extracted from a plurality of feature maps, or identify an error in at least one of the plurality of feature maps by using the plurality of feature maps and estimate a state related to a disease of a subject based on at least one feature map excluding the at least one feature map in which an error has been identified. Since utilization of a plurality of feature maps increases information used for estimation and/or enables use of information with few errors, the precision of estimation can be enhanced.

A state of a subject estimated through the processing 700 is provided to, for example, a physician, and the physician can utilize this as an indicator for diagnosis. Since a state of a subject estimated through the processing 700 is estimated in accordance with a feature map that can have knowledge of a specialist or expert integrated therein, precision and reliability can be high.

The processing 700 can further comprise step S703 and step S704 for analyzing which classification among a plurality of secondary clusters contributes to the state estimated at step S702.

At step S703, the survival time analyzing means 143 of the processing unit 140 analyzes the survival time of a subject based on a feature map. The survival time analyzing means 143 can analyze the survival time of a subject by using, for example, Kaplan-Meier method, log-rank test, Cox proportional hazards model, etc.

At step S704, the identifying means 144 of the processing unit 140 identifies at least one secondary cluster contributing to an estimated state of a subject among a plurality of secondary clusters in a feature map from a result of analyzing the survival time at step S703. For example, the identifying means 144 can identify a secondary cluster with a high hazard ratio (e.g., secondary cluster with the highest hazard ratio, secondary cluster having a hazard ratio equal to or greater than a predefined threshold value, etc.) obtained from analyzing the survival time at step S703 as a secondary cluster contributing to an estimated state.

A factor unique to a subject having a disease with poor prognosis such as usual interstitial pneumonia can be identified by analyzing what the factor contributing the estimated state is in this manner. Such a factor can be utilized as an indicator for diagnosis. This can lead to accurate and simple diagnosis.

The aforementioned examples with reference to FIGS. 5, 6, and 7 describe that processing is performed in a specific order, but the order of each processing is not limited to those that are described. Processing can be performed in any theoretically possible order.

The aforementioned examples with reference to FIGS. 5, 6, and 7 describe that the processing of each step shown in FIGS. 5, 6, and 7 is materialized by the processing unit 120, processing unit 130, or processing unit 140 and a program stored in the memory unit 150, but the present invention is not limited thereto. At least one of processing of each step shown in FIGS. 5, 6, and 7 may be materialized by a hardware configuration such as a control circuit. Alternatively, at least one of each step shown in FIGS. 5, 6, and 7 may be performed by a human using a computer system or measuring instrument.

The aforementioned examples describe an example in which the system 100 is implemented as a server, but the present invention is not limited thereto. The system 100 can also be implemented by any information terminal apparatus (e.g., terminal apparatus 300).

The aforementioned examples describe that a feature map is outputted by using a machine learning model, but a machine learning model outputted by the system 100 of the invention is not limited to a machine learning model dedicated for feature maps. The system 100 can be utilized for creating a machine learning model for classification. The system 100 can create a machine learning model that is capable of outputting a meaningful classification, even for data other than an image, by causing an initial machine learning model to learn any data for learning other than images. This can be achieved by the same processing as the processing 500 described above, except for a plurality of images for learning being a plurality of data for learning.

For example, genetic sequence data can be utilized as data for learning. In such a case, the reclassifying means 123 preferably receives a user input from a specialist or expert of genetics and reclassifies in accordance therewith. A machine learning model created in this manner can classify inputted genetic sequence data in a genetically meaningful classification.

For example, pathological report data can be utilized as data for learning. In such a case, the reclassifying means 123 preferably receives a user input from a specialist or expert of pathology and reclassifies in accordance therewith. A machine learning model created in this manner can classify inputted pathological report data in a meaningful classification as a pathological report.

The aforementioned examples describe that a state related to a disease of a subject is estimated by using a feature map, but the system 100 of the invention can also estimate any other state. For example, a therapeutic effect in view of a medical treatment (e.g., surgery, drug dosing, etc.) can be determined, or overall survival in view of a medical treatment (e.g., surgery, drug dosing, etc.) can be predicted.

The present invention is not limited to the embodiments described above. It is understood that the scope of the present invention should be interpreted based solely on the claims. It is understood that an equivalent scope can be practiced based on the descriptions of the present invention and common general knowledge from the specific descriptions in the preferred embodiments of the invention.

### Examples

### (Creation of an Initial Machine Learning Model)

A WSI from tissue staining was scanned at a magnification of 20× by using an Aperio CS2 scanner (Leica Biosystems). The WSI contained images from 53 subjects (31 males, 22 females, average age of 59.57 (standard deviation of 11.91)) having a disease of the interstitial pneumonia family (IPF/UIP, rheumatoid arthritis, systemic sclerosis, diffuse alveolar damage, pleuroparenchymal fibroelastosis, organizing pneumonia, or sarcoidosis symptom). The WSI was fragmented into images of 280×280 pixels at 2.5× resolution, 5× resolution, and 20× resolution.

An initial machine learning model was created by self-supervised learning with fragmented images with each of 2.5× resolution, 5× resolution, and 20× resolution by using 151 WSIs. A CNN (ResNet18) that outputs a feature consisting of a 128-dimensional vector is utilized as the base of an initial machine learning model.

At this time, each image was randomly flipped over or rotated between 0° to 200 to expand the data for learning. Furthermore, each image was randomly cut out to a size of 244×244 to match the original dimension of ResNet18.

### (Clustering)

Fragmented images with each of 2.5× resolution, 5× resolution, and 20× resolution were inputted into an initial machine learning model by using 151 WSIs, and each image was quantified into a 128-dimensional vector. Clustering was performed for each 128-dimensional vector by k-means method for classification into a respective initial cluster of a plurality of initial clusters.

### (Reclassification)

Images classified into an initial cluster were presented to two pathologists, and the pathologists reclassified the images into pathologically meaningful secondary clusters.

### (Creation of a Machine Learning Model)

Transfer learning was performed by fine tuning a CNN of an initial machine learning model by using results of reclassification. At this time, not only the weighting of a fully connected layer, but also parameters of previous layer were optimized.

### (Use of a Machine Learning Model)

WSIs from 182 lung biopsy cases was inputted into the machine learning model described above, and a feature map was created based on the resulting classification.

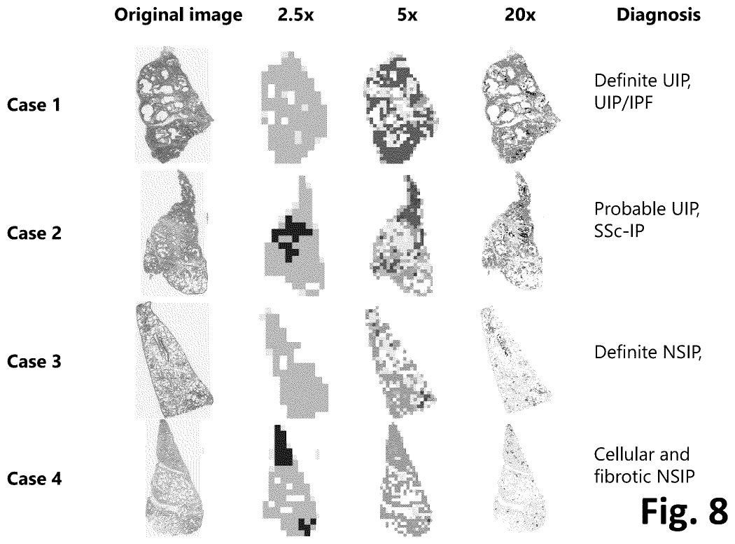

FIG. 8 shows an example of the results thereof.

FIG. 8 shows inputted WSIs, feature maps created in accordance with an output from a machine learning model created at a 2.5× resolution, feature maps created in accordance with an output from a machine learning model created at a 5× resolution, and feature maps created in accordance with an output from a machine learning model created at a 20× resolution. A physician was asked to diagnose a disease of a subject from these feature maps.

In case 1, a subject was diagnosed as Definite UIP and UIP/IPF from the feature maps.

In case 2, a subject was diagnosed as Probable UIP and SSc-IP from the feature maps.

In case 3, a subject was diagnosed as Definite NSIP from the feature maps.

In case 4, a subject was diagnosed as Cellular and fibrotic NSIP from the feature maps.

### (UIP Diagnosis 1)

Outputs of the machine learning model described above were used to estimate whether the disease is UIP based on a plurality of findings (secondary clusters) contained in a feature map created at a 5× resolution. As a Comparative Example, whether a disease is UIP was estimated based on results of clustering outputs from an initial machine learning model. The number of clusters in clustering was varied to 4, 8, 10, 20, 30, 50, and 80 to estimate whether the disease was UIP in each case. Estimation used random forest.

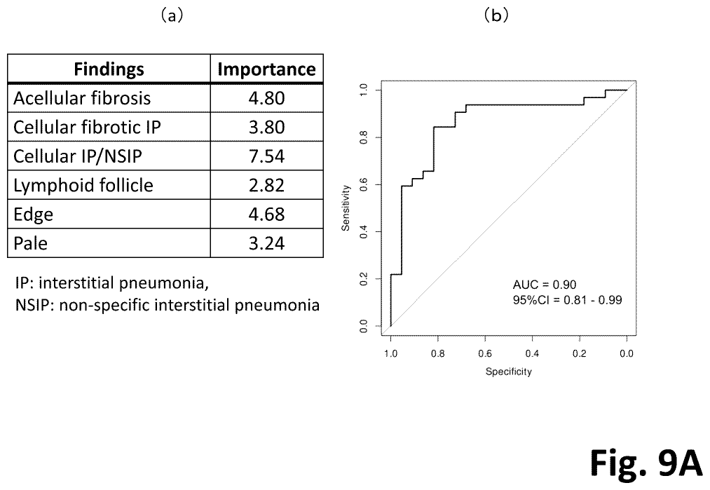

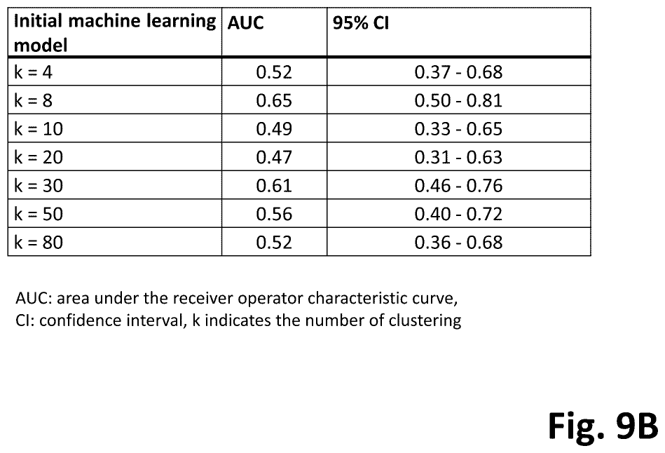

FIG. 9A shows results of estimation based on an output of the machine learning model described above, and FIG. 9B shows results of estimation based on an output of an initial machine learning model.

FIG. 9A (a) is a table showing results of calculating the importance of each feature in random forest, wherein importance of each finding (secondary cluster) for UIP prediction is shown. This example shows that findings (secondary clusters) of “Cellular IP/NSIP” and “Acellular fibrosis” are important for estimating whether a disease is UIP.

FIG. 9A (b) shows a ROC curve (Receiver Operating Characteristic curve). AUC (Area Under the Curve) represents the precision of estimation and had a high value of 0.90.

In FIG. 9B, AUC was at best 0.65 (for number of clusters of 8) in estimating through an output from an initial machine learning model. It can be understood that the precision of estimation based on an output of the machine learning model described above was significantly higher than the precision of estimation based on an output of an initial machine learning model.

### (UIP Diagnosis 2)

Whether a disease is UIP was estimated by using a feature map created at a 2.5× resolution, a feature map created at a 5× resolution, and a feature map created at a 20× resolution individually and combinations thereof, based on a plurality of findings (secondary clusters) contained in each feature map.

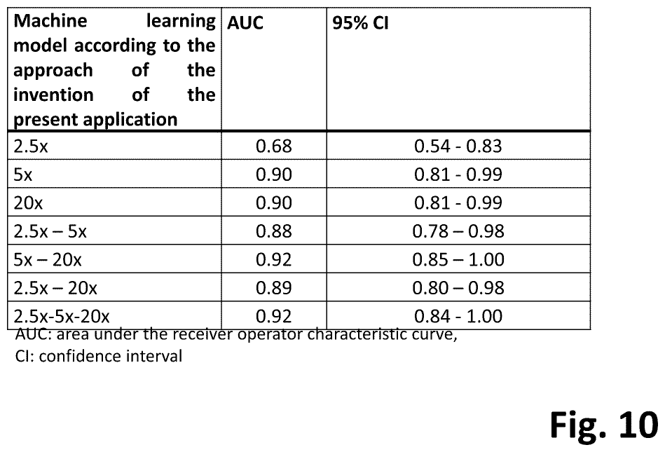

FIG. 10 shows the results thereof. AUC was 0.68 in UIP estimation using a feature map created at a 2.5× resolution. AUC was 0.90 in UIP estimation using a feature map created at a 5× resolution. AUC was 0.90 in UIP estimation using a feature map created at a 20× resolution. AUC was 0.88 in UIP estimation using a feature map created at a 2.5× resolution and a feature map created at a 5× resolution. AUC was 0.92 in UIP estimation using a feature map created at a 5× resolution and a feature map created at a 20× resolution. AUC was 0.89 in UIP estimation using a feature map created at a 2.5× resolution and a feature map created at a 20× resolution. AUC was 0.92 in UIP estimation using a feature map created at a 2.5× resolution, a feature map created at a 5× resolution, and a feature map created at a 20× resolution. It can be understood in this manner that precision was high in each case, except when using a feature map created at a 2.5× resolution alone. It can be understood that the precision was higher than the precision of estimation based on an output of an initial machine learning model shown in FIG. 9B.

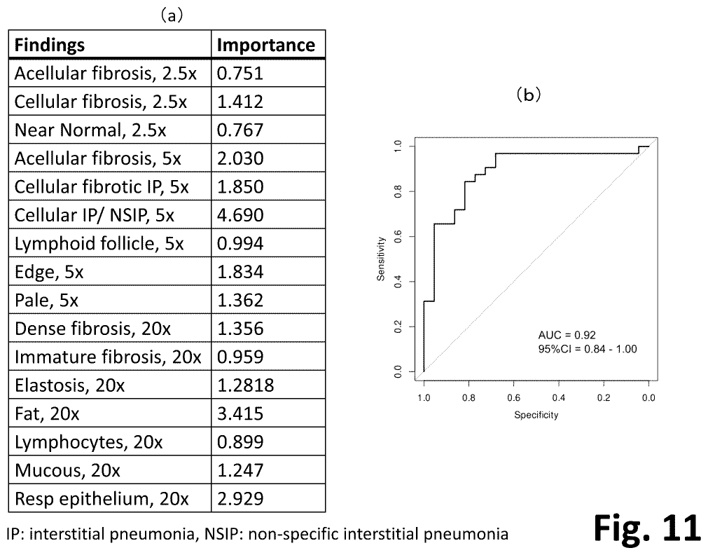

FIG. 11 shows results of UIP estimation by using a combination of a feature map created at a 2.5× resolution, a feature map created at a 5× resolution, and a feature map created at a 20× resolution.

FIG. 11(a) is a table showing results of calculating the importance of each feature in random forest, wherein importance of each finding (secondary cluster) for UIP prediction is shown. This example shows that findings (secondary clusters) of “Cellular IP/NSIP” and “Fat” are important for estimating whether a disease is UIP.

FIG. 11(b) shows a ROC curve (Receiver Operating Characteristic curve). AUC (Area Under the Curve) had a high value of 0.92, as shown in FIG. 10.

### (Analysis of Overall Survival)

All of a feature map created at a 2.5× resolution, a feature map created at a 5× resolution, and a feature map created at a 20× resolution were used to calculate a hazard ratio (HR) of each finding (secondary cluster) for overall survival. A Cox proportional hazards model was used for the calculation.

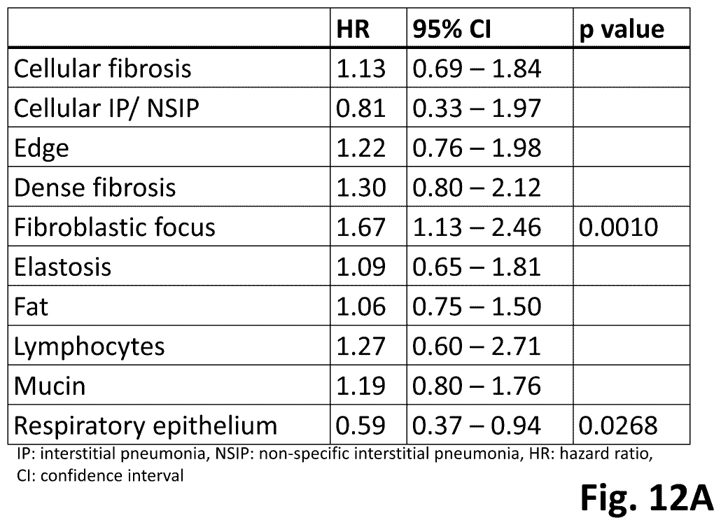

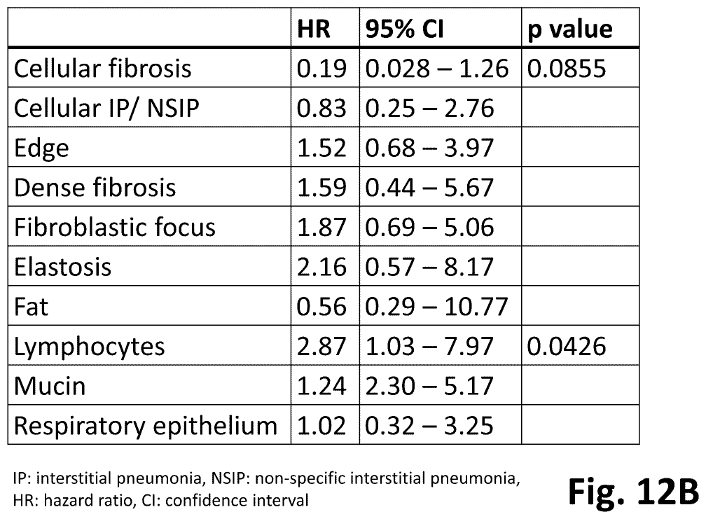

FIG. 12A shows results of calculation using a Cox proportional hazards model for cases that have been diagnosed as UIP by a pathologist. This example has shown that the finding (secondary cluster) of “Fibroblastic focus” is a factor for poor prognosis. Specifically, it was shown that prognosis is likely poor with a finding of “Fibroblastic focus” in a subject diagnosed as UIP.

FIG. 12B shows results of calculation using a Cox proportional hazards model for cases that were not diagnosed as UIP by a pathologist. This example shows that the finding (secondary cluster) of “Lymphocytes” is a factor for poor prognosis. Specifically, it was shown that prognosis is likely poor with the finding of “Lymphocytes” in a subject who was not diagnosed as UIP.

In this manner, if a feature map created by the machine learning model of the invention is used, various analyses can be performed, and the precision of diagnosis can be improved by using this in diagnosis.

### (Application to CT Image)

A machine learning model was created by creation of an initial machine learning model, clustering, and reclassification by using CT images of a lung.

A lung field region was extracted in a high resolution CT image obtained from 60 interstitial pneumonia patients and a 32 pixel×32 pixel patch therein was obtained. Self-supervised learning was performed on a patch obtained in this manner to obtain a feature extractor optimized for CT images of interstitial pneumonia.

By using the resulting feature extractor, a plurality of initial clusters were obtained from converting patches of the same 60 cases to features and clustering the features. Tiles were able to be efficiently labeled by an interstitial pneumonia specialist integrating the initial clusters and reorganizing the clusters into medically significant findings. A machine learning model for classifying patches to findings corresponding to plurality of secondary clusters based on the labeling was constructed.

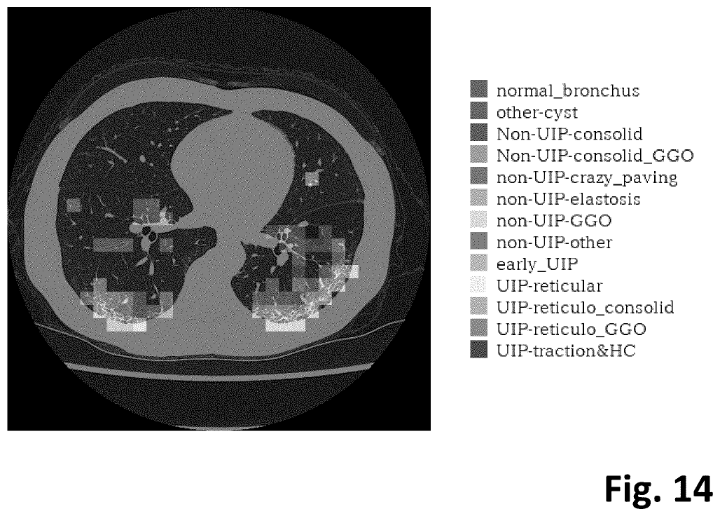

FIG. 14 shows an example of inputting a high resolution CT image of a lung field region into the machine learning model constructed in this manner.

As can be understood from FIG. 14, local findings in CT were able to be classified into a medically meaningful finding by applying the machine learning model of the invention to a high resolution CT image of a lung field region.

### INDUSTRIAL APPLICABILITY

The present invention is useful for providing a machine learning model that is capable of integrating human knowledge.

### REFERENCE SIGNS LIST

100 system
110 interface unit
120, 130, 140 processing unit
150 memory unit
200 database unit
300 terminal apparatus
400 network

1.-26. (canceled)

27. A method of creating a machine learning model comprising:
receiving a plurality of data for learning;
classifying each of the plurality of data for learning into a respective initial cluster of a plurality of initial clusters by using an initial machine learning model, the initial machine learning model being caused to at least learn to output a feature of an inputted datum from the datum;
reclassifying the plurality of initial clusters into a plurality of secondary clusters based on the plurality of data for learning classified into the respective plurality of initial clusters; and
creating a machine learning model by causing the initial machine learning model to learn a relationship between the plurality of initial clusters and the plurality of secondary clusters, the machine learning model classifying an inputted datum into one of the plurality of secondary clusters.

28. The method of claim 27, wherein the reclassifying comprises:
presenting the plurality of data for learning classified into the respective plurality of initial clusters to a user;
receiving a user input that associates each of the plurality of initial clusters to one of the plurality of secondary clusters; and
reclassifying the plurality of initial clusters into a plurality of secondary clusters based on the user input.

29. The method of claim 28, wherein the plurality of secondary clusters are defined by the user.

30. The method of claim 27, wherein the plurality of secondary clusters are determined in accordance with a resolution of the plurality of data for learning.

31. The method of claim 27, wherein the plurality of data is a plurality of images.

32. The method of claim 31, wherein the plurality of images for learning comprise at least one of a plurality of partial images from fragmenting an image at a predefined resolution, an image for a pathological diagnosis, an image of tissue of a subject with interstitial pneumonia, an image of tissue of a subject without interstitial pneumonia, or a plurality of images of subjects with different diseases.

33. The method of claim 27 further comprising repeating the receiving, the classifying, and the reclassifying of datum within at least one secondary cluster among the plurality of secondary clusters as the plurality of data for learning.

34. The method of claim 27, wherein the created machine learning model is used for outputting a feature map.

35. A method of creating a machine learning model, comprising:
receiving a plurality of data classified into at least one secondary cluster by a machine learning model created in accordance with the method of claim 27;
classifying each of the plurality of received data into a respective initial cluster of a plurality of initial clusters by using an initial machine learning model, the initial machine learning model being caused to at least learn to output a feature of an inputted datum from the datum;
reclassifying the plurality of initial clusters into a plurality of secondary clusters based on the plurality of received data classified into the respective plurality of initial clusters; and
creating a machine learning model by causing the initial machine learning model to learn a relationship between the plurality of initial clusters and the plurality of secondary clusters, the machine learning model classifying an inputted datum into one of the plurality of secondary clusters.

36. A method of creating a feature map, comprising:
receiving a target image;
fragmenting the target image into a plurality of regional images;
classifying each of the plurality of regional images into a respective secondary cluster of the plurality of secondary clusters by inputting the plurality of regional images into a machine learning model created by the method of claim 31; and
creating a feature map by separating each of the plurality of regional images in the target image in accordance with respective classifications.

37. The method of claim 36, wherein the separating comprises coloring regional images belonging to the same classification among the plurality of regional images with the same color.

38. A method of estimating a state related to a disease of a subject, comprising:
obtaining a feature map created in accordance with the method of claim 36, the target image being an image of tissue of the subject; and
estimating a state related to a disease of the subject based on the feature map.

39. The method of claim 38, wherein the estimating the state comprises at least one of estimating a type of interstitial pneumonia of the subject or estimating whether the subject has usual interstitial pneumonia.

40. The method of claim 38, wherein the estimating a state related to a disease of the subject based on the created feature map comprises:
calculating a frequency of each of the plurality of secondary clusters from the feature map; and
estimating a state related to the disease based on the frequency.

41. The method of claim 38, wherein creating the feature map comprises creating a plurality of feature maps, the plurality of feature maps having different resolutions from one another.

42. The method of claim 41, wherein the estimating a state related to a disease based on the created feature map comprises:
calculating a frequency of each of the plurality of secondary clusters from each of the plurality of feature maps; and
estimating a state related to the disease based on the frequency.

43. The method of claim 41, wherein the estimating a state related to a disease based on the created feature map comprises:
identifying an error in at least one of the plurality of feature maps by using the plurality of feature maps; and
estimating a state related to the disease based on at least one feature map excluding the at least one feature map in which an error has been identified.

44. The method of claim 38, further comprising:
analyzing survival time of the subject whose state related to the disease has been estimated based on the created feature map; and
identifying at least one secondary cluster contributing to the estimated state among a plurality of secondary clusters in the feature map.

45. A system for creating a machine learning model, comprising:
receiving means for receiving a plurality of data for learning;
classifying means for classifying each of the plurality of data for learning into a respective cluster of a plurality of initial clusters by using an initial machine learning model, the initial machine learning model being caused to at least learn to output a feature of an inputted datum from the datum;
reclassifying means for reclassifying the plurality of initial clusters into a plurality of secondary clusters based on the plurality of data for learning classified into the respective plurality of initial clusters; and
creating means for creating a machine learning model by causing the initial machine learning model to learn a relationship between the plurality of initial clusters and the plurality of secondary clusters, the machine learning model classifying an inputted datum into one of the plurality of secondary clusters.

46. A computer readable storage medium for storing a program for creating a machine learning model, the program being executed in a computer system comprising a processing unit, the program causing the processing unit to execute processing comprising:
receiving a plurality of data for learning;
classifying each of the plurality of data for learning into a respective cluster of a plurality of initial clusters by using an initial machine learning model, the initial machine learning model being caused to at least learn to output a feature of an inputted datum from the datum;
reclassifying the plurality of initial clusters into a plurality of secondary clusters based on the plurality of data for learning classified into the respective plurality of initial clusters; and
creating a machine learning model by causing the initial machine learning model to learn a relationship between the plurality of initial clusters and the plurality of secondary clusters, the machine learning model classifying an inputted datum into one of the plurality of secondary clusters.
